(19) United States
(12) Patent Application Publication
(10) Pub. No.: US 2023/0048804 A1
(43) Pub. Date: Feb. 16, 2023

(54) SYSTEMS AND METHODS FOR ENHANCING LEARNING VIA TRANSCUTANEOUS AURICULAR VAGUS NERVE STIMULATION

(71) Applicant: Texas Christian University, Fort Worth, TX (US)

(72) Inventor: Tracy M. Centanni, Fort Worth, TX (US)

(73) Assignee: Texas Christian University, Fort Worth, TX (US)

(21) Appl. No.: 17/809,695

(22) Filed: Jun. 29, 2022

Related U.S. Application Data
(60) Provisional application No. 63/202,927, filed on Jun. 30, 2021.

(51) Int. Cl. A61N 1/36; G09B 19/06

(52) U.S. Cl. CPC A61N 1/36036; A61N 1/36034; G09B 19/06; A61N 1/0456

(57) ABSTRACT

Systems and methods for increasing learning via transcutaneous auricular vagus nerve stimulation is presented. The disclosure can include positioning a device within a human ear and delivering specifically timed and modulated pulses of electrical energy to the auricular vagus nerve transcutaneously, avoiding invasive procedures commonly associated with vagus nerve stimulation. The present disclosure can enhance reading comprehension, facilitate the learning letter-sound relationships, and provide for increased retention of new languages for a patient, all without surgically implanting a device into a patient.

### CROSS-REFERENCE TO RELATED APPLICATIONS

The present application claims priority from an earlier filed provisional application, Ser. No. 63/202,927, filed Jun. 30, 2021, with the same title and by the same inventor.

### BACKGROUND

### Field of the Disclosure

The field of the disclosure relates generally to enhancing learning of individuals, such as in linguistics and literacy, via stimulation of the auricular vagus nerve, especially in a non-invasive manner.

### Background of the Disclosure

The ability to communicate is a critical skill, whether through spoken language or through print. While humans are born with the capacity for language, it becomes significantly more difficult to learn a new language with age. Learning to read is similarly challenging in adulthood and for those with developmental disorders, yet reading is also a critical skill in modern society. Expert reading acquisition is marked by fluent, effortless decoding and adequate comprehension skills and is required for modern daily life. In spite of its importance, many individuals struggle with reading comprehension even when decoding skills are adequate. Unfortunately, effective reading comprehension interventions are also limited, especially for adults. While the ability to acquire a novel language and attain fluency in that language is clearly beneficial, it is significantly more difficult to acquire such skills in adulthood. Many adults are nevertheless required to learn a new orthography for personal or vocational reasons, but while traditional in-person and computer training programs can aid in this process, learning is often slow and retention is quite poor—few, if any, result in native-like fluency.

Little is known about biologically-based interventions for language learning, with few examples in the literature at all, and those examples pose several inherent problems. For example, while some studies have focused on the use of surgically-implantable cervical vagus nerve stimulation devices in an attempt to drive neural plasticity, such methods are extremely invasive and not practical for reading intervention. Therefore, a method for driving long-lasting neural plasticity during language learning would be valuable for those who need or want to achieve fluency in a novel language later in life.

### SUMMARY OF THE DISCLOSURE

In one embodiment, the present disclosure can include non-invasive transcutaneous auricular vagus nerve stimulation (taVNS) to enhance novel orthography acquisition in individuals, including children, young adults, adults, and the elderly. For example, 37 typically developing participants, randomly assigned to a computer control, device sham control, earlobe stimulation control, or experimental transcutaneous auricular stimulation (taVNS) group in accordance with principles of the present disclosure. In one example, participants then learned novel letter-sound correspondences in Hebrew over five training lessons. In another example, Performance was assessed using three measures to evaluate various aspects of reading: Letter ID, Automaticity, and Decoding. In one embodiment, the taVNS group significantly outperformed the three control groups on both the Automaticity and Decoding tasks. In another embodiment, there was no difference on the Letter ID task. In one example, these results demonstrate, for the first time, that taVNS is capable of improving aspects of reading acquisition in adults. These findings have potential implications for a wide range of cognitive tasks.

In another embodiment, the present disclosure can include transcutaneous auricular vagus nerve stimulation (taVNS) paired with training that is capable of improving learning and retention of words in a new language as compared to sham stimulation. For example, twenty-nine typically developing young adults completed a one-hour training session in which they learned 30. Palauan nouns while receiving sham, 5 Hz, or 25 Hz stimulation to the left posterior tragus. In one embodiment, participants completed a free-recall translation test immediately after training and seven days later to quantify learning and retention. In another embodiment, while there was no effect of stimulation group on translation test performance immediately after training, there was an effect at retention. In another embodiment, seven days after training, the 25 Hz taVNS group significantly outperformed both the sham and 5 Hz taVNS groups, with no difference between the sham and 5 Hz taVNS groups. In one example, these results suggest that taVNS may improve retention of novel vocabulary words and that stimulation frequency may impact efficacy. In one embodiment, frequency of the stimulation can impact efficacy; in another embodiment, frequency of the stimulation may have little to no effect on efficacy.

In another embodiment, a growing body of research suggests that non-invasive transcutaneous stimulation of the auricular vagus nerve (taVNS) may drive neural plasticity for low-level reading skills such as speech sound perception and letter-sound learning. Thus, in another embodiment, the present disclosure can include taVNS paired with passage reading can improve performance on a higher order reading skill, such as comprehension. In another embodiment, twenty-four typically developing young adults were recruited and screened for baseline reading and working memory skills. In another embodiment, participants received either sham or active taVNS while reading short passages out loud. In another embodiment, immediately following each passage, participants answered a series of comprehension questions that required either direct recall of passage details or inference based on passage content. In another embodiment, while taVNS did not improve the mechanics of reading (e.g., reading rate or accuracy), there was a significant benefit of active taVNS on reading comprehension. In another embodiment, this effect was driven by significant improvement on accuracy for memory-based questions while there was no effect of taVNS on inference question accuracy. In one example, these results suggest that taVNS may improve performance on memory-based tasks, but not on inferential processes. In another example, these findings suggest that taVNS may be beneficial for enhancing memory, but its efficacy may be limited in higher cognitive domains.

### BRIEF DESCRIPTION OF THE DRAWINGS

The present disclosure will be readily understood by the following detailed description, taken in conjunction with the accompanying drawings that illustrate, by way of example, the principles of the present disclosure. The drawings illustrate the design and utility of one or more exemplary embodiments of the present disclosure, in which like elements are referred to by like reference numbers or symbols. The objects and elements in the drawings are not necessarily drawn to scale, proportion, or precise positional relationship. Instead, emphasis is focused on illustrating the principles of the present disclosure.

FIG. 1 illustrates exemplary participant characteristics and assessment scores by group, in accordance with one or more embodiments of the present disclosure;

FIG. 2A illustrates one exemplary electrode location on participants' ears for the device sham and taVNS groups as described in the present disclosure;

FIG. 2B shows another exemplary electrode location on participants' ears for the earlobe stimulation group, as further described in the present disclosure;

FIG. 3 illustrates taVNS thresholding measurements in accordance with one or more embodiments of the present disclosure, the average of four measurements being used in subsequent training sessions and checked for comfort each day;

FIG. 4 illustrates Hebrew letters and pronunciations learned over a five-day training period in accordance with one or more embodiments of the present disclosure;

FIG. 5A illustrates an exemplary structure of a training program in accordance with one or more embodiments of the present disclosure in which participants learned 1-2 new letters;

FIG. 5B is similar to FIG. 5A, showing participants' practice of all combinations of letters learned to date;

FIG. 5C is similar to FIG. 5B, but showing the end of the learning session where the participants completed word-like practice;

FIG. 6A illustrates performance on three measures across control groups in accordance with one or more embodiments of the present disclosure, with FIG. 6A showing no effect of control conditions on Letter ID;

FIG. 6B is similar to FIG. 6A, but shows performance effect on automaticity;

FIG. 6C is similar to FIG. 6B, but shows effect on performance decoding;

FIG. 7A illustrates an exemplary effect of taVNS on outcome measures showing no effect of taVNS on Letter ID due to ceiling effect;

FIG. 7B is similar to FIG. 7A but showing that taVNS significantly improved speed on Automaticity tasks;

FIG. 7C is similar to FIG. 7B, but showing taVNS significantly improved percent correct on decoding tasks;

FIG. 8 illustrates correlations between six English reading measures and the three Hebrew assessments in control group participants (n=23) in accordance with one or more embodiments of the present disclosure;

FIG. 9 illustrates correlations between six English reading measure and three Hebrew assessments in the taVNS group (n=14) in accordance with one or more embodiments of the present disclosure;

FIG. 10 illustrates participant characteristics and standard assessment scores (M±SD) by experimental group in accordance with one or more embodiments of the present disclosure;

FIG. 11 illustrates exemplary taVNS thresholding measurements in accordance with one or more embodiments of the present disclosure;

FIG. 12 illustrates exemplary trained Palauan words with corresponding English translations in accordance with one or more embodiments of the present disclosure;

FIG. 13 illustrates descriptive statistics (M±SEM) for outcome measures during training and on the free-recall translation assessment in accordance with one or more embodiments of the present disclosure;

FIGS. 14A and 14B illustrate part of an exemplary procedure for exposure and knowledge checks during training in accordance with one or more embodiments of the present disclosure, where FIGS. 14A and B illustrate participants seeing a Palauan word for 4 seconds before seeing the next word;

FIGS. 14C and 14D represent a knowledge check in which participants saw a Palauan word with four English translations and selected the correct one;

FIGS. 15A and 15B illustrate exemplary data from knowledge checks during training in accordance with one or more embodiments of the present disclosure with FIG. 15A showing no effect of the stimulation group on accuracy during training and FIG. 15B showing no effect on time to respond to a knowledge check;

FIG. 16A illustrates exemplary performance on Palau-to-English translation test after training and at retention in accordance with one or more embodiments of the present disclosure with FIG. 16A showing that there was no effect of the stimulation group on performance after training;

FIG. 16B is similar to FIG. 16A, but showing no effect of the stimulation group seven days after training;

FIG. 17 illustrates current intensity against performance at retention showing current intensity does not correlate with performance at retention;

FIGS. 18A-18F illustrate an exemplary relationship between verbal learning core and performance by stimulation group at various stimulation frequencies, suggesting that there were no relationships between performance on the Verbal Learning Core Measure and performance on free-call translation at post training within the sham taVNS;

FIGS. 19A-19F are similar to FIGS. 18A-18F in illustrating an exemplary relationship between verbal learning recall and performance by stimulation group using Pearson's correlations;

FIG. 20 illustrates exemplary participant characteristics and standard assessment scores (M±SD) by stimulation group in accordance with one or more embodiments of the present disclosure;

FIGS. 21A and 21B illustrate an example of passage and associated questions, respectively, administered during reading session in accordance with one or more embodiments of the present disclosure;

FIGS. 22A and 22B illustrate taVNS effect on reading mechanics in accordance with one or more embodiments of the present disclosure, showing no difference between active taVNS and sham taVNS on average error during reading or reading rate, respectively;

FIG. 23A illustrates exemplary performance on reading comprehension questions in accordance with one or more embodiments of the present disclosure, with FIG. 23A showing a significant benefit of active taVNS compared to sham taVNS on comprehension;

FIG. 23B is similar to FIG. 23A, showing the effect was driven by a significant benefit of active taVNS for memory based comprehension questions;

FIG. 23C is similar to FIG. 23B, showing the same effect was not true for interference-based comprehension questions;

FIG. 24 illustrates an exemplary relationship between current intensity and performance on memory-based comprehension questions in participants receiving active taVNS in accordance with one or more embodiments of the present disclosure; and

FIGS. 25A and 25B illustrate exemplary classifications of comprehension questions as memory-based or inference-based by passage in accordance with one or more embodiments of the present disclosure.

### DETAILED DESCRIPTION OF THE DISCLOSURE

The preferred version of the disclosure presented in the following written description and the various features and advantageous details thereof, are explained more fully with reference to the non-limiting examples included in the accompanying drawings and as detailed in the description, which follows. Descriptions of well-known components have been omitted so to not unnecessarily obscure the principle features described herein. The examples used in the following description are intended to facilitate an understanding of the ways in which the disclosure can be implemented and practiced. Accordingly, these examples should not be construed as limiting the scope of the claims.

### EXAMPLE #1

### Introduction

Reading is a critical skill for modern life, as daily communication relies on print. The development of the brain's reading network is a protracted process, requiring many years of practice, lasting into early adulthood. The sensitive period for reading closes around the age of 18-19, possibly due to the long trajectory for reading network acquisition and the amount of practice needed to achieve expertise. One marker of expertise is fluency, the ability to read and comprehend a word without decoding individual letters. Although adults learning to read in a novel orthography may achieve a level of speed that allows for comprehension, they may never achieve native-like fluency. In spite of this obstacle, there are many situations in which an adult may need to achieve native-like fluency in a new orthography. Some examples include business professionals needing to review documents during international meetings, subsequent generations of immigrant families wanting to read historical scriptures, and military officers needing to communicate with local residents during deployment and times of critical events. Prior research on literacy programs suggests that adults can learn to read when provided adequate training, but the learning time is long and retention performance is poor. It is therefore clear that current behavioral programs are insufficient to induce long-term fluency. Thus, the goal of the current study was to evaluate a novel method for re-opening the brain's sensitive window for orthography learning.

One established method involves stimulating the vagus nerve to activate the nucleus tractus solitarius (NTS), which has projections to the nucleus basalis (NB) and locus coeruleus (LC). Together, they release key neurotransmitters important for driving brain plasticity and learning and memory: acetylcholine and norepinephrine, respectively. In mice, stimulation of NE was found to aid in long term potentiation for an extended period of time and stimulation of the LC aided rats in an auditory perception task. Further, disruption of norepinephrine release in rats blocked plasticity driven by vagus nerve stimulation (VNS). These regions are also tied to learning in humans, as higher LC activation is associated with improved memory on a delayed gratification task. VNS allows for targeted release of norepinephrine and acetylcholine without invasive direct brain stimulation (i.e., deep brain stimulation), providing easier access to this neural plasticity mechanism in patient populations.

Cervical vagus nerve stimulation (cVNS) is FDA approved for the treatment of epilepsy and depression and is in clinical trials for stroke and tinnitus. This approach involves surgically implanting a cuff electrode around the vagus nerve, located in the neck, and a pulse generator positioned subcutaneously below the clavicle or axilla. Pairing the timing of cVNS with an external stimulus (e.g., sound or movement) drives long-lasting and meaningful neural plasticity. For example, cVNS paired with a specific tone drives sensory plasticity in primary auditory cortex (A1) of a rat, specific to the frequency of the paired tone. This approach led to a novel tinnitus treatment, now in clinical trials. cVNS is also capable of driving plasticity in the motor cortex when paired with specific movements both in the rat model and in clinical trials with patients experiencing upper limb motor deficits.

In the cognitive domain, cVNS, has increased performance on tasks relying on working memory. For example, delivering cVNS after paragraph reading improved recognition of highlighted words. In another study, participants receiving cVNS had decreased error rates during a delayed recall task. cVNS also improved performance on digit-symbol and verbal fluency tasks. Together, results demonstrate that cVNS can increase performance and decrease error rates in cognitive tasks, suggesting it may also aid in other cognitive tasks, such as reading.

In spite of the success with cVNS, such an invasive and expensive procedure is not a practical intervention for cognitive skills like reading. The auricular branch of the vagus nerve (ABVN) projects to the outer ear and can be accessed at either the cymba conchae region of the pinna or the posterior surface of the tragus. fMRI studies have demonstrated that transcutaneous auricular vagus nerve stimulation (taVNS) activates similar medullary and deep brain structures as cVNS, without the need for an invasive surgery. Growing evidence suggests that taVNS may also provide a comparable neural plasticity effect compared to cVNS. For example, taVNS improved rehabilitation of post-stroke motor function recovery in humans and increased performance on a memory task in older adults. Given the comparable success of taVNS and cVNS on improving motor control after stroke and improving cognitive tasks, we hypothesized that taVNS paired with training would significant improve orthography acquisition in five days.

### Methods

### Participants

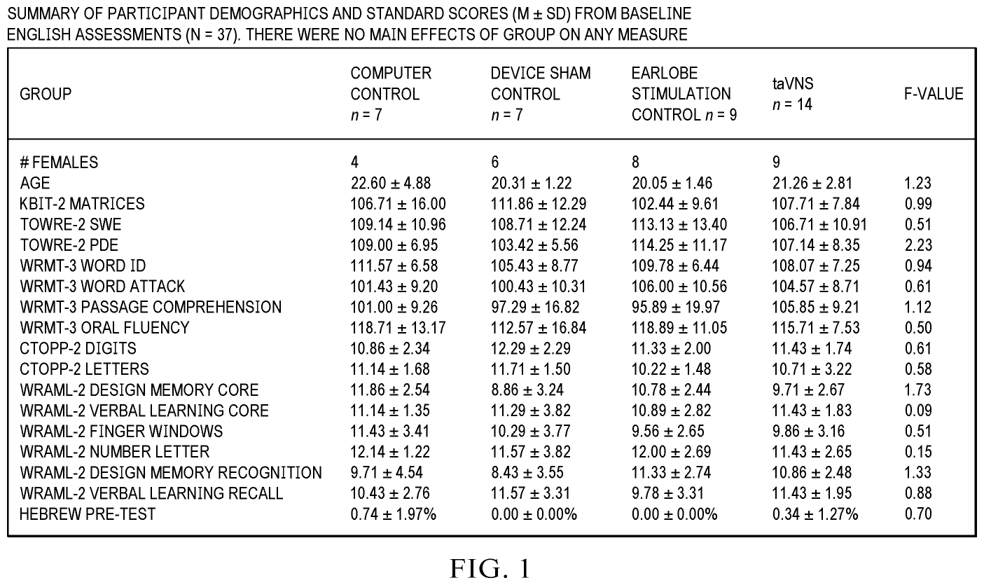

In total, 122 participants were screened for eligibility, with 37 participants meeting these criteria. To be eligible for the study, participants needed to: (a) be a native English speaker, (b) be between the ages of 18 and 35, (c) achieve a standard score of 85 or higher on the KBIT-2 Matrices, (d) achieve standard reading scores of 90 or higher on each of the four measures described below, (e) have no history of neurological disorders, diagnoses, or medications, (f) have no medical implants, and (g) not been previously exposed to Hebrew or a language of similar orthography. We screened 122 individuals, but 4 were excluded for a low IQ score, 44 for low reading scores on one or more measures, 8 for exclusionary medications, 8 for medical implants, procedures, or diagnoses, 2 for previous exposure to a similar orthography, 3 for being outside of the age range, and 14 for issues in scheduling or withdrawing from the study. Two participants were trained but errors were made during administration of outcome assessments. Thus, our final sample included 37 participants. A summary of participant demographics and standard scores (mean±SD) from baseline English assessments (N=37) (there were no main effects of group on any measure) can be seen in FIG. 1. are provided in FIG. 1. The protocol was approved by the Texas Christian University Institutional Review Board, and participants provided written informed consent prior to enrollment.

Participants were assessed using a background survey and a standardized battery to ensure they were fluent readers in their native English. The battery included the matrices subtest of the KBIT-2, as a measure of nonverbal IQ, as well as four reading measures: the Sight Word Efficiency and Phonemic Decoding Efficiency subtests of the TOWRE-2 and the Word ID and Word Attack subtests of the WRMT-3. In addition to these eligibility measures, we administered additional assessments, including the passage comprehension and oral fluency subtests of the WRMT-3, rapid automatized naming (RAN) of digits and letters (CTOPP-2), and working memory and attention subtests from the WRAIVIL-2. A second researcher reviewed all scoring, and discrepancies were resolved by a consensus between both researchers. Participants also completed a brief Hebrew Letter ID pre-test to confirm no prior knowledge of to-be-learned letters. All participants scored less than 5% accuracy, with no group differences (FIG. 1).

taVNS Device Settings

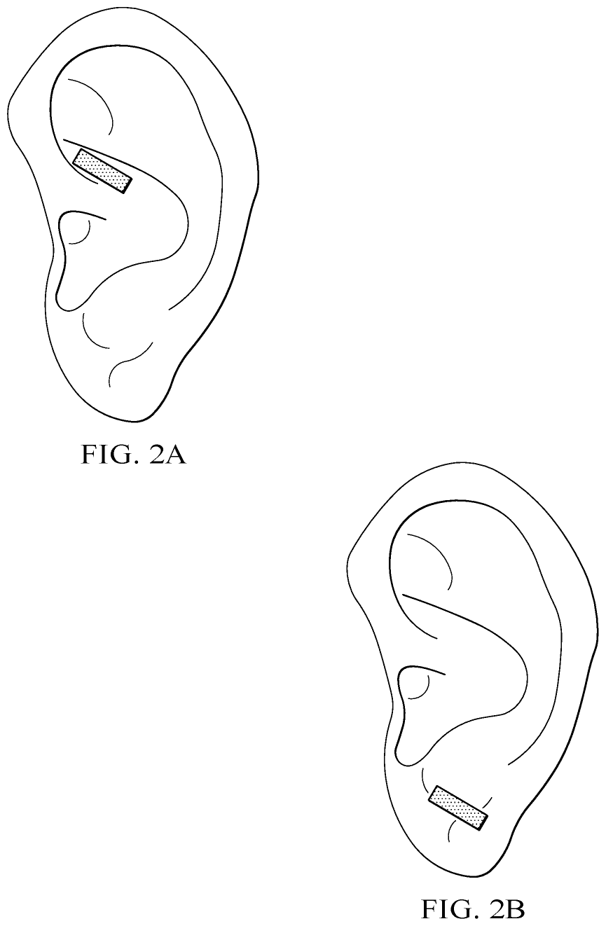

FIGS. 2A and 2B are examples of electrode location in groups wearing an exemplary taVNS device. For example, the earpiece was placed on participants' left ear at location shown by the grey bar. For the device sham and taVNS groups, the electrode was placed at the cymba ocncha region of the left ear (e.g., FIG. 2A). For the earlobe stimulation control group, the electrode was placed at the earlobe of the left ear (e.g., FIG. 2B). Prior to placing the auricular neurostimulation device on the left ear (e.g., FIGS. 2A and 2B), participants cleaned the skin on and around the ear with an alcohol wipe to remove any excess oils and ensure optimal conductivity. Conductive hydrogels were placed on the earpiece to control current flow and ensure participant comfort. A 1-cm long cylindrical stimulating electrode was placed either at the left cymba conchae or the left earlobe, depending on group assignment. There are well-demonstrated differences in the effect of stimulating the left versus right branches of the vagus. The right branch is connected to the sinoatrial node, making this branch more effective at driving cardiac change. To ensure activation of the NTS, which is critical for neural plasticity, and to avoid cardiac change, we chose to stimulate only the left ear. The earlobe was used as a control location as fMRI studies demonstrated stimulation of the earlobe does not activate the NTS. The current return electrode (1 cm×3 cm) was located behind the ear over the mastoid bone. Current was delivered as a square, biphasic pulse, at 5 Hz, and a 200 μs pulse width. These parameters and electrode placement were chosen based on prior work. Stimulation intensity was determined for each participant individually, as described below. The device was controlled by custom Python programming to ensure precise timing of the stimulation.

taVNS Group Assignment and Thresholding

Eligible participants were randomized into one of four experimental groups: computer control, device sham control, earlobe stimulation control, or experimental taVNS. The computer control group (n=7) participants completed the automated training program without any interaction with or knowledge of the stimulator. The remaining participants went through a taVNS thresholding procedure, wore the earpiece, and were told they would receive stimulation during training. To account for placebo effects or beliefs about wearing the earpiece, the device sham control group (n=7) wore the earpiece at the left cymba concha (the same anatomical location as the taVNS group) and was told stimulation would occur, but the device was turned off without the participants' knowledge. All sham participants were told that our thresholding procedure was designed to determine a comfortable stimulation intensity for each individual, and that each person may experience that current differently. At the end of the study, no participants reported being suspicious of their stimulation group.

To determine whether the sensation of stimulation anywhere on the left ear increased performance, the earlobe stimulation control group (n=9) wore the device and received active stimulation to the left earlobe, as earlobe stimulation does not activate the NB or LC. Finally, the taVNS group (n=14) wore the earpiece during all training sessions and received stimulation to the left cymba concha.

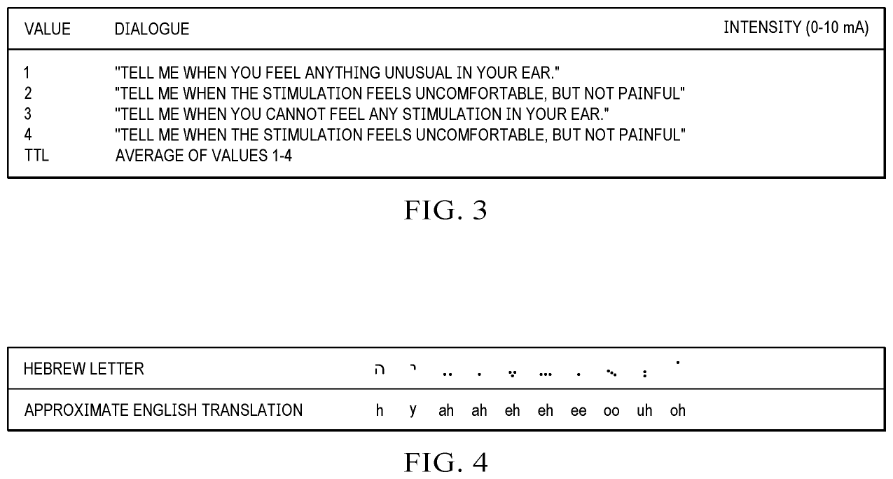

All participants who wore the device, regardless of the electrode's location, completed a thresholding procedure in which a trained researcher determined a customized amount of current for each participant. Thresholding took place in the location the stimulator would be worn. To determine comfortable current for each participant, we obtained two measurements at minimum threshold and two at the upper level of comfort (FIG. 3). Thresholding procedure used with all participants in the device sham control, earlobe stimulation control and experimental taVNS groups. Four measurements were acquired during thresholding for each participant. The average of these four measurements was used in subsequent training sessions and checked for comfort each day. The upper level of comfort was defined as the point when stimulation became distracting and uncomfortable but prior to the onset of pain. The average of these four measurements was calculated and used as the current intensity setting during the training sessions. There were no group differences between the device sham control (2.04±1.18 mA), earlobe stimulation control (1.51±0.35 mA), and taVNS groups (1.68±0.87 mA) in thresholding current intensity (F (2, 27)=0.81, p=0.46).

### Training Program

Eligible participants returned to the lab on five separate days for 30-min training sessions, which were conducted individually in sound-dampened testing rooms in the lab. We chose to train participants over several days to ensure we could measure higher-order reading skills, such as decoding. Lesson length was modeled after commonly used orthography training programs such as DuoLingo. At the beginning of each session, a brief test was conducted to ensure that the participant's customized level of thresholding current was comfortable. Then, the participant completed a self-paced lesson, presented through custom PsychoPy programming. All instructions and feedback were provided by a pre-recorded, female native-English speaker. Participants were instructed to practice reading letters out loud and point along to the letters both during practice as well as during feedback. A trained researcher was always present in the room to ensure participant safety and compliance with instructions. No adverse events occurred during training.

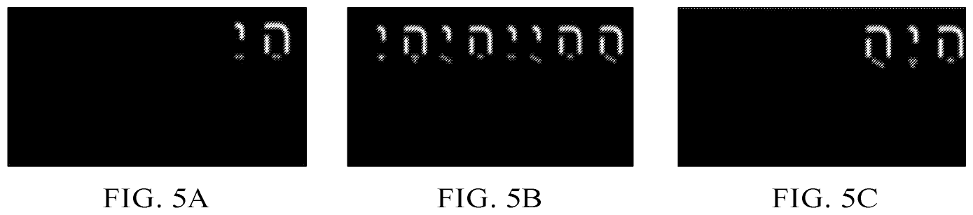

Training lessons were structured in a uniform manner and were designed to mimic best practices for new orthography learning in adults. Each training lesson began with a review of the letters learned on previous days. Next, one or two new letters were introduced and practiced individually (FIG. 5A)—following review of previously learned graphemes, participant learned 1-2 new letters. For example, the vowel sound “eh,” shown in FIG. 5A by two dots below the consonant, in the context of previously earned consonants ( [symbol] and [symbol] ). This was followed by practice in series of letters (FIG. 5B) where participants practiced all combinations learned to date in a series by reading from right to left. For certain trials, font size and angle were varied to improve generalizability of learned letters. Participants verbally sounded out the sequence of letters and pressed a button when finished. After the participant read the series out loud, the correct responses to the same sequence were presented in the auditory domain while the participant pointed along, ensuring attention to the feedback. Those in the earlobe stimulation control and taVNS groups received stimulation during this multi-sensory auditory and visual feedback to ensure that only correct pairings were reinforced. Stimulation lasted approximately 6-8 s per sequence and occurred during approximately 215 letter-sound pairings per session. Finally, each training session concluded with participants practicing sequences of letters arranged as real or pseudowords, with the instruction to blend the sounds together (FIG. 5C). As in series practice, stimulation was only paired with feedback. After five training lessons, participants had learned two consonants and eight vowels in Hebrew using closely approximated English phonemes (FIG. 4). Sixteen letter-sound correspondences were taught over the course of the five training days. Two consonants (h and y) were taught on the first day, and vowels were added each day throughout the training.

### Hebrew Assessments

After five lessons, participants were assessed on their knowledge of trained Hebrew graphemes through three assessments: Letter ID, Automaticity, and Decoding. These measures were generated in-house and based on standard English assessments. During the Letter ID task, participants were presented with sixteen consonant-vowel (CV) combinations, presented individually, and instructed to provide the correct sound, with no time pressure. This measure was based on the Letter ID task from the WRMT-3. An incorrect answer earned 0 points, a partially correct answer (i.e., getting the consonant or vowel correct, but not both) earned 0.5 points, and a correct answer earned 1 point. Scores were added together and converted to a percentage, where a higher score indicated better performance.

The Automaticity task was based on the RAN subtest of the CTOPP-2. Participants saw an eight-by-four grid of Hebrew CV combinations. The participant sounded out every CV combination on the entire card as quickly and accurately as possible. A researcher timed the task, rounded the time to the nearest second, and added a 1 s penalty per error made. Thus, better performance was indicated by faster times on this task.

Finally, the Decoding task was based on the Phonemic Decoding Efficiency subtest of the TOWRE-2. Participants viewed a card of pseudowords written in Hebrew and read through the list as quickly and accurately for 45 s. Performance on this measure was scored as percent correct. Higher performance was indicated by a higher percent correct.

### Statistical Analysis

A one-way ANOVA was used to evaluate whether there were effects of control condition on performance across the three dependent measures. No group differences were found, so control groups were combined to test our a priori hypothesis that taVNS would improve performance on letter-sound learning using one-tailed independent-samples t-tests. Descriptive statistics for all outcome measures are presented as mean±SEM.

Additionally, we conducted analyses to evaluate the relationships between English reading measures and performance on Hebrew outcome assessments. Spearman correlations (rs) were used for the Letter ID task, as participants exhibited a ceiling effect, and Pearson's correlations (r) were used for the Automaticity and Decoding tasks. The Bonferroni correction was used to control for multiple comparisons within each set of correlations comparing English assessments to a single outcome measure (6 comparisons per set).

### Results

taVNS Improves Novel Orthography Acquisition

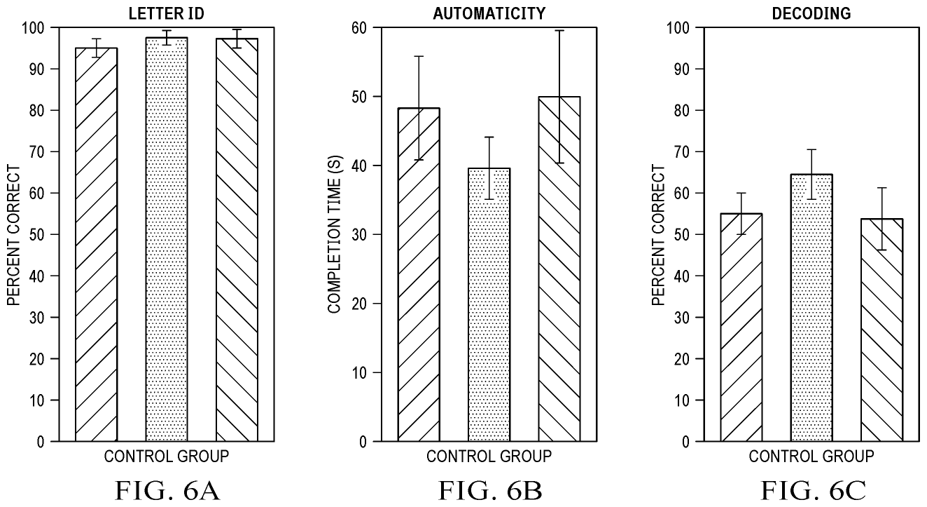

Thirty-seven participants completed training and outcome assessments. We first evaluated performance across the control groups to determine whether there was evidence of a placebo effect in any of these conditions. There was no significant main effect of group for the Letter ID task (F (2, 20)=0.43, p=0.65; FIG. 6A), the Automaticity task (F (2, 19)=0.52, p=0.60; FIG. 6B) or the Decoding task (F (2, 19)=0.83, p=0.45; FIG. 6C). The control groups were therefore combined for all subsequent analyses (see Supplement for additional analyses).

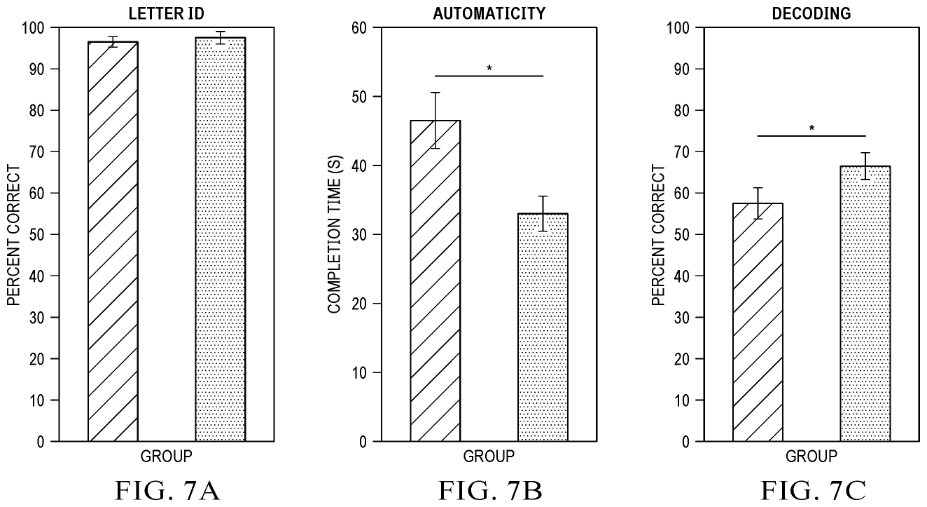

There was no significant difference in Letter ID performance between the combined control group (96.51±1.20%) and the taVNS group (97.47±1.44%; t (35)=0.52, p=0.30; FIG. 7A). On the Automaticity task, the taVNS group completed the task significantly faster (33.14±2.32 s) than the combined control group (46.27±4.34 s; t (34)=2.28, p=0.014; FIG. 7B). On the Decoding task, the taVNS group had a higher percent correct (66.45±3.68%) than the combined control group (57.49±3.14%; t (34)=1.72, p=0.048; FIG. 7C).

### Correlations Between English and Hebrew Reading Measures

Secondary analyses were then conducted to examine the relationship between the six English reading measures (Sight Word Efficiency, Phonemic Decoding Efficiency, Word Identification, Word Attack, Rapid Digit Naming, and Rapid Letter Naming), administered in the initial assessment session and the Hebrew outcome assessments administered after training.

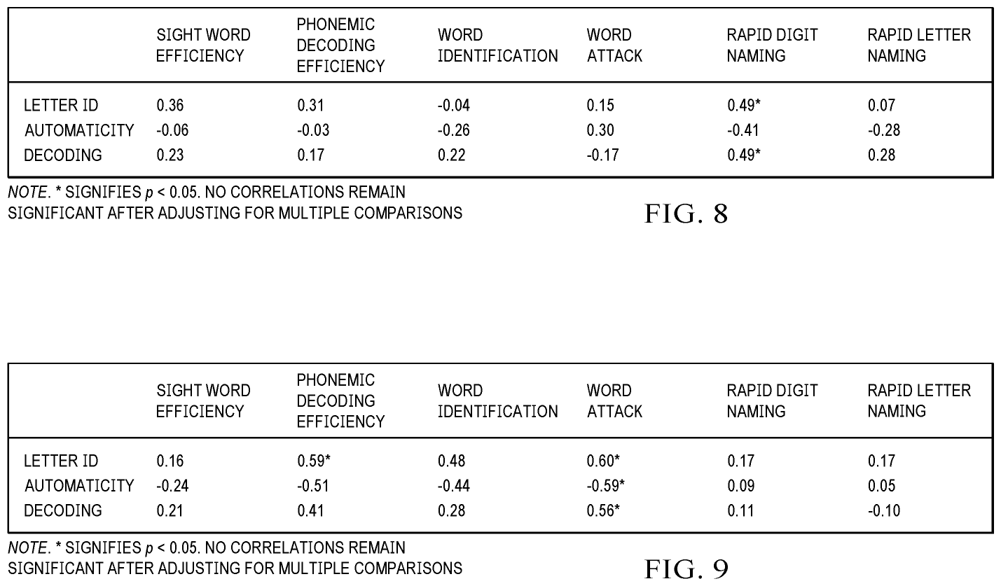

Across all control group participants (n=23), there were nominally significant positive relationships between Rapid Digit Naming with Hebrew Letter ID (rs=0.49, p=0.02) and Hebrew Automaticity (r=0.49, p=0.02), such that faster digit naming times were related to a higher percent correct on identifying Hebrew letters and on a timed decoding task. None of these comparisons survived correction (FIG. 8).

To determine whether English measures were predictive of benefits conferred by taVNS, we evaluated the same relationships in the taVNS group alone (n=14). There was a nominally significant relationship such that higher scores on the Phonemic Decoding task in English were related to a higher percent correct on identifying Hebrew letters (rs=0.59, p=0.03). In these analyses, the English Word Attack measure significantly correlated with all of the Hebrew assessments, such that higher scores on an untimed pseudo word task was related to a nominally higher percent correct on identifying Hebrew letters (rs=0.60, p=0.02), faster times on a Hebrew Automaticity task (r=—0.58, p=0.03), and a higher percent correct on the Hebrew Decoding task (r=0.56, p=0.04). No comparisons survived correction (FIG. 9).

### Discussion

In the current study, we tested the hypothesis that taVNS paired with novel letter-sound correspondence training in Hebrew would improve performance on outcome measures. We observed a significant effect of taVNS paired with training on both Automaticity and Decoding, with no effect on Letter ID. These findings support the hypothesis that taVNS is effective at improving letter-sound learning in young adults.

### Relationships Between Native and Novel Orthographies

Baseline measures of reading in young children, such as rapid naming and phoneme awareness measures, are predictive of future reading abilities. In the current study, we found various nominally significant correlations between baseline English assessments and post-training Hebrew assessments.

Across the control groups, there were nominally significant positive relationships between rapid digit naming and Letter ID and Decoding in the novel orthography. RAN is a common measure of naming speed and is associated with future reading outcomes in children. Thus, it is not surprising that in our sample, individuals with better rapid automatized naming in English performed better on tasks in the novel orthography. It is interesting to note, however, that the relationship was limited to digits, perhaps suggesting that in the short time window of training, novel letters were processed more like symbols than letters. Early in reading acquisition, letter symbols are processed largely by right hemisphere regions as objects, with a leftward lateralization occurring only with practice. The brain's reading network is not hard-wired and develops with practice and instruction. The brain may therefore need more practice than provided in our training in order for the VWFA to process a novel orthography as print rather than as symbols. When other symbols, such as houses and faces are used as a system of print, ten training sessions were used to evoke activation in the left VWFA for trained versus untrained stimuli. In another embodiment, training programs can be conducted over a longer trajectory to determine whether novel orthography symbols are ever processed in the same brain region as native orthographies.

In the subset of participants receiving taVNS, a different pattern of relationships emerged. Interestingly, the relationships between rapid digit naming and the novel orthography measures were no longer significant. Instead, timed decoding was significantly correlated with Hebrew Letter ID, and performance on an untimed pseudo word reading measure (Word Attack) was significantly correlated with every outcome measure. The Word Attack measure in English requires knowledge of the letters, an automatic connection between the letter and the phoneme, and the ability to blend sounds together to decode non-words. It is interesting that the rapid naming measures were not correlated with outcomes in the taVNS group. If RAN scores predict learning of a novel orthography using purely behavioral methods as discussed above, it may suggest that such training approaches encourage the brain to follow the same trajectory as in initial reading acquisition, with novel letters processed as symbols prior to being recognized as print. The lack of relationship between RAN and outcome measures in the taVNS group suggest that taVNS may push the brain to bypass this process and instead take advantage of existing left-hemisphere circuits already well suited for the task at hand. In another embodiment, techniques, such as neural imaging, can be used to determine whether taVNS accelerates the normal trajectory for orthography acquisition or pushes the brain to use existing circuits more effectively.

### Applications for Non-Invasive Stimulation

The ability to read fluently in a novel orthography is increasingly important in the modern developed world. Well-known and highly used programs, such as Rosetta Stone and DuoLingo are useful in second language learning and contain an orthography component, but the effects are dubious. The addition of a non-invasive stimulation component may improve orthography learning in typical readers, as our results demonstrate a significant benefit of taVNS on orthography learning in five days.

In some countries, many individuals never acquire reading as children and are therefore learning to read for the first time as adults. Despite efforts by many international organizations to generate evidence-based literacy programs, most individuals never achieved native-like fluency, and a lack of practice leads to regression into illiteracy. taVNS devices are small and portable and may be useful additives to literacy programs in regions of the world that are difficult to access. In the current study, all participants were well-educated, native readers in English, so it is unknown whether this approach will be effective in novice adults or struggling readers. In another embodiment, this approach may be effective in illiterate adults.

### Limitations

There are three main limitations of the current study. First, while the results were robust, a small sample size recruited from a pool of undergraduate students stunts the ability to generalize findings. In another embodiment, the present disclosure can include a larger group of individuals, to account for effects of gender, varied backgrounds and occupations, and a range of baseline reading abilities. In another embodiment, taVNS interacts with neurotransmitters like norepinephrine, acetylcholine, and serotonin. We chose a stimulation frequency based on previous research in epilepsy, migraine, and anti-inflammatory models which demonstrated that lower stimulation frequencies (1-10 Hz) were more effective at activating associated neural structures than higher stimulation frequencies (20-30 Hz). However, VNS may also be effective at other current intensities or frequencies, including 25 Hz [30, 31, 32], or 30 Hz, as well as in subthreshold conditions. Our effect may be muted by the choice of a lower stimulation frequency. In another embodiment, the present disclosure can include frequency optimization and a comparison of stimulation at and below sensory threshold. Third, we did not investigate whether the addition of taVNS improves retention of learned relationships after training ends. For taVNS to be relevant for the general public, the effects must be long-lasting.

### EXAMPLE #2

### Introduction

Although language acquisition is relatively easy for children, it becomes significantly more difficult after puberty, with extensive training required for adults to reach fluency (Johnson & Newport, 1989; Lenneberg, 1967; Long, 1990, but see Birdsong & Molis, 2001; Hartshorne, Tenenbaum, & Pinker, 2018). Although both children and adults are able to learn novel pseudowords, children exhibit better retention than adults, even after a short delay (Bishop, Barry, & Hardiman, 2012), supporting the idea that plasticity for second language (L2) acquisition decreases across the lifespan. In spite of the increased difficulty, learning of a second language is often important for older children and adults for a variety of personal and vocational reasons. For example, families moving abroad may enroll their children in local schools, where knowledge of a new language is required. Further, military professionals are frequently deployed to an unfamiliar region on short notice. Being familiar with keywords in the local language is advantageous for personnel to both communicate and successfully navigate the region. There is also evidence that language acquisition can improve one's career income (McManus, Gould, & Welch, 1983) and overall health (Bialystok, Craik, & Luk, 2012), including a potential neuroprotective effect of bilingualism (Bialystok, Craik, & Freedman, 2007, but see Van den Noort et al., 2019). Given an increased need for L2 acquisition and fluency in adulthood, more efficient and successful methods for improving vocabulary retention are needed. In the current study, we investigated the use of a novel approach to enhance learning and retention of novel vocabulary words in young adults.

Current approaches to second language learning consist largely of in-person classroom options, one-on-one tutoring, and automated computer programs (e.g., DuoLingo and Rosetta Stone; Lu, 2008), using strategies such as recall and restudy (Krishnan, Watkins, & Bishop, 2017). While in-person coursework is a common choice for adolescents and young adults, more adults are turning to computer programs and apps for their language learning needs. For example, DuoLingo reported a large user base of 300 million active users in 2020 (duolingo.com). In spite of the popularity of this approach, the time required to make progress is significant and the effectiveness of this method is impacted by motivation to study consistently (Loewen et al., 2019). Even in those with a high degree of motivation, the retention of learned material after studying ends is quite poor (Nielson, 2011). One study investigated retention via a recall and recognition test in teaching Spanish-English word pairs and found that recall tests were harder than recognition tests, and shorter intersession intervals led to worsened performance compared to longer intersession intervals (Bahrick & Phelps, 1987). This increased difficulty in novel language acquisition for older children and adults may be due to a variety of factors (for a review, see Birdsong, 2018) including decreasing plasticity with age (Hurford, 1991), changes in myelination (Long, 1990), age-related cognitive decline (Lee, 2004), or variable differences in education and motivation (Vanhove, 2013). Regardless of the reason, data suggest that failures in language learning are likely due to poor retention of learned information rather than a deficit in initial learning. Although performance on vocabulary recognition tends to be high immediately after training (Brown et al., 2012), performance significantly decreases as the testing delay increases (e.g., Salavati & Salehi, 2016). Thus, improvement of retention is critical for designing more effective language learning programs.

Poor retention may be driven by a number of cognitive factors related to developmental plasticity changes and baseline skills in the native language that impact the degree to which words in the novel language are retained. Working memory (e.g., Kim, 2017; for a review, see Archibald, 2017) and short-term memory (Majerus, 2013) skills have both been evaluated in vocabulary learning and retention. Working and short-term memory play important roles in encoding and consolidation, since associations between the written and verbal parts of a word need to be correctly stored for access in the future. For example, working memory skills are significantly correlated with performance on word learning and retrieval tasks (Hazrat, 2015), and performance on a letter/digit span task is a strong predictor of L2 learning rate (Atkins & Baddeley, 1998). This suggests that various memory systems work together to increase vocabulary learning and retention, and verbal short-term memory may be a stronger predictor than verbal working memory in children learning L2 (Verhagen & Leseman, 2016). Thus, if memory skills are critical for language learning, a method for reliably and quickly improving verbal memory would be beneficial in a wide array of language learning applications.

One promising method for driving such long-lasting neural plasticity is vagus nerve stimulation (VNS). A growing body of evidence suggests that stimulation of the afferent branch of the vagus nerve drives significant and long-lasting neural plasticity in sensory (Engineer et al., 2011), motor (Khodaparast et al., 2013; Porter et al., 2012), and cognitive (Clark et al., 1999; Sackeim et al., 2001; Sun et al., 2017) domains. Stimulation of this nerve is possible non-invasively by targeting the auricular branch of the vagus nerve (ABVN) at either the cymba concha (Redgrave et al., 2018; Thakkar et al., 2020) or the posterior side of the tragus (Badran et al., 2018). Stimulation of the ear at these locations leads to increased activation in deep brain structures shown to be critical for effective vagus nerve stimulation (Frangos, Ellrich, & Komisaruk, 2015; Yakunina, Kim, & Nam, 2016). taVNS paired with training drives significant neural plasticity in a variety of domains, including post-stroke motor function recovery (Redgrave et al., 2018) and tinnitus (Yakunina, Kim, & Nam, 2018). In the cognitive domain, taVNS paired with training increased performance on a face-name association task (Jacobs et al., 2015), during speech sound category learning (Llanos et al., 2020), and during novel letter-sound learning (Thakkar et al., 2020). Previous studies have successfully utilized a variety of stimulation frequencies, including 1 Hz (Straube et al., 2015), 5 Hz (Thakkar et al., 2020), 8 Hz (Jacobs et al., 2015), 10 Hz (Stefan et al., 2012), or 25 Hz (Llanos et al., 2020; Redgrave et al., 2018; Ylikoski et al., 2017). Vagus nerve stimulation may therefore be effective in aiding the learning and retention of vocabulary words in a novel language.

The current study was designed to answer two specific research questions. First, we investigated if taVNS is capable of improving learning and retention of novel words in young adults. Second, given the range of stimulation intensities utilized in the literature, we investigated whether stimulation frequency impacts efficacy. We evaluated the efficacy of sham versus low (5 Hz) or high (25 Hz) frequency taVNS on young adults' ability to learn and retain vocabulary words in a novel language.

### Methods

### Participants

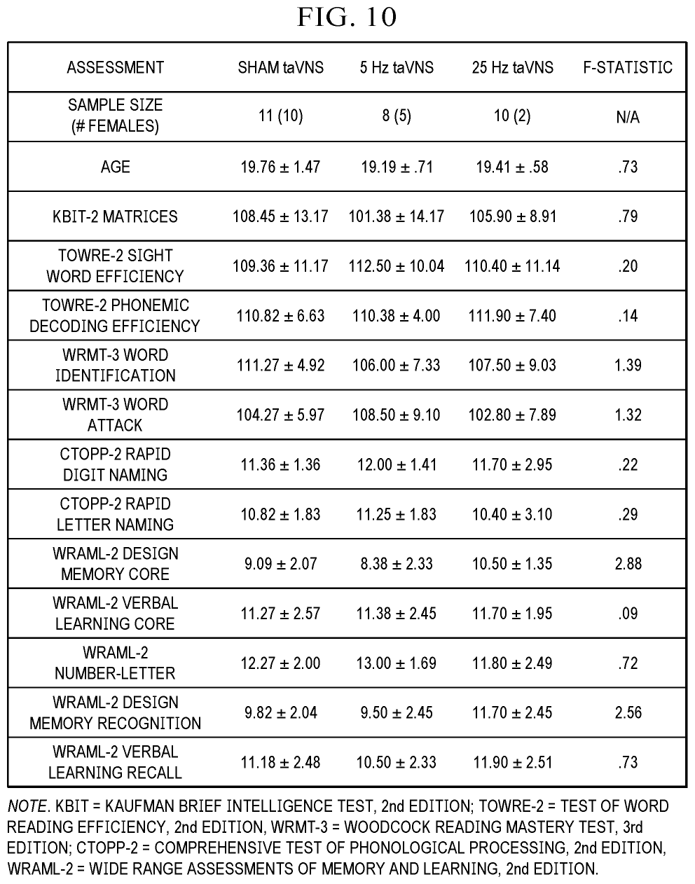

We recruited and screened 84 undergraduate students using an initial one-hour eligibility session. This screening session included a short online survey about personal history of reading and motor development, diagnoses, medications, and family history, and assessment on five standardized assessments of IQ and reading. The matrices subtest of the KBIT-2 (Kaufman & Kaufman, 2004) was utilized as a measure of nonverbal IQ. To evaluate baseline reading ability, we administered the Sight Word Efficiency and Phonemic Decoding Efficiency subtests of the TOWRE-2 (Torgesen, Wagner, & Rashotte, 2012) and the Word Identification and Word Attack subtests of the WRMT-3 (Woodcock, 2011). Since the training program required participants to read both English and Palau words on a screen with a time limit, we excluded participants with low baseline reading scores. To be eligible for the study (i.e., a typically developing young adult), participants had to: (a) be a native English speaker, (b) be between the ages of 18 and 35, (c) have a standard score of 85 or higher on nonverbal IQ, (d) have standard scores of 90 or higher on each of the four reading measures listed above, (e) have no history of diagnoses, medications, disorders, or implants, and (f) have no prior experience with the trained language (Palau). Of the 84 participants who were screened for eligibility, two participants were excluded for low IQ, 23 for low reading scores, 19 for exclusionary medications or diagnoses, and four for issues in scheduling or withdrawing from the study. Seven additional participants were unable to complete the study due to the onset of the COVID-19 pandemic. Thus, the final sample included 29 typically developing young adults (FIG. 10).

A number of descriptive assessments were also administered but were not used to determine eligibility. These additional assessments included the Rapid Digit Naming and Rapid Letter Naming subtests of the CTOPP-2 (Wagner, Torgesen, & Rashotte, 2013) and the Design Memory Core, Verbal Learning Core, Number-Letter, Design Memory Recognition, and Verbal Learning Recall subtests of the WRAML-2 (Sheslow & Adams, 2009). Two researchers scored each assessment independently and resolved discrepancies in scoring by discussion and consensus. Final raw scores were then converted to age-normed standard scores. The study was approved by the Texas Christian University Institutional Review Board, and all participants provided written informed consent prior to beginning the study.

taVNS Device and Procedures

Transcutaneous auricular vague nerve stimulation (taVNS) was administered using the ParaSym device (https://www.parasym.co/index.html), which delivers current through a small, circular, ¼ inch in diameter gold-plated copper electrode, placed at the posterior tragus. This stimulation location was chosen to ensure activation of the auricular branch of the vagus nerve, which innervates the nucleus tractus solitarius (NTS; Badran et al., 2018; Yakunina, Kim, & Nam, 2016). In the two active stimulation groups, current was delivered as square, biphasic pulses with 200 μs pulse width. Stimulation frequency varied by group assignment and was either set at 5 Hz stimulation (n=8) or at 25 Hz stimulation (n=10). In the sham stimulation group (n=11), the device was turned off without participant knowledge. Stimulation was delivered to the left outer ear, as prior VNS research has suggested that stimulation of the right branch results in more bradycardia than does stimulation of the left branch (see Butt et al., 2020; Yuan & Silberstein, 2015, for reviews).

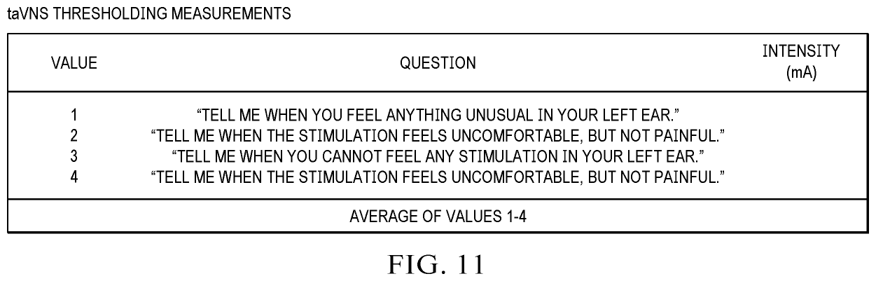

All participants, regardless of group assignment, completed a thresholding procedure to determine the appropriate amount of current for each individual prior to the training session. To determine this customized current level, a trained researcher recorded two measurements at the participant's absolute minimum threshold of sensation and two measurements at the upper end of comfort, prior to the onset of pain (Thakkar et al., 2020; Yakunina, Kim, & Nam, 2016; FIG. 11). Thresholding was completed using 5 Hz for the sham taVNS and 5 Hz taVNS groups, while the 25 Hz taVNS group was evaluated at 25 Hz. Thresholding current intensities were significantly lower in the 25 Hz taVNS group (1.96±0.13 mA) compared to the 5 Hz taVNS group (2.33±0.11 mA; two-tailed, unpaired t-test: t (16)=2.22, p=0.04).

### Palau Vocabulary Training Program

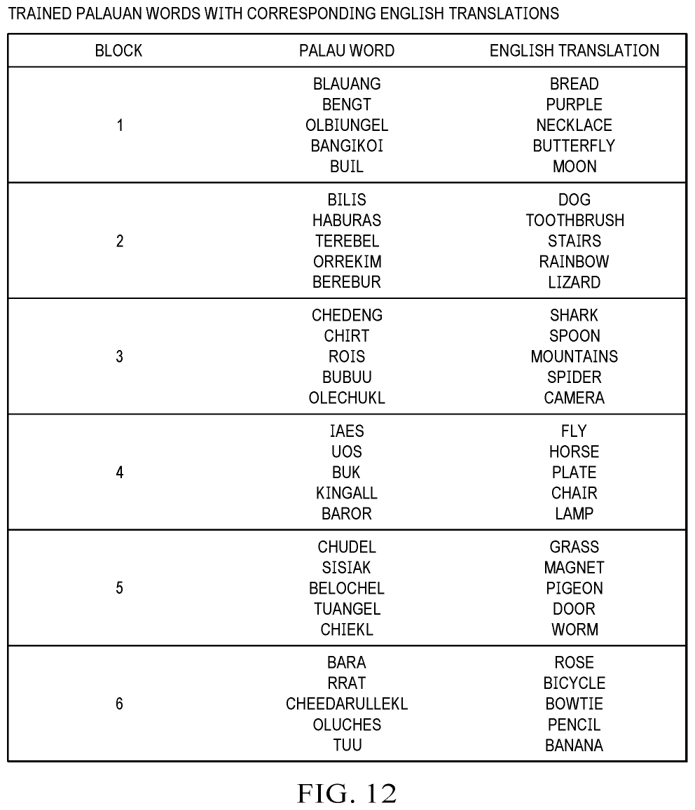

Eligible participants completed a 1-hour training session in which they learned 30 novel vocabulary words in Palau, a language which utilizes the Latin alphabet (FIG. 12). Since the training program included visual text and images without any auditory stimuli, we chose the training language so as to avoid any confounds caused by learning a language with an unfamiliar orthography. To ensure no prior knowledge with the training set, participants completed a brief free-response translation pre-test of the Palauan words used in this study. All participants in all groups scored 0% accuracy on this task. The training program was designed to mimic the presentation of novel vocabulary in existing commercial programs, such as DuoLingo (Duolingo, Pittsburgh, Pa.; https://www.duolingo.com/) and Rosetta Stone (Rosetta Stone, Arlington, Va.; https://www.rosettastone.com/), with alternating blocks of exposure and knowledge checks. Stimulation was turned on during exposure blocks and turned off during knowledge checks and was manually controlled by a trained researcher from behind a barrier.

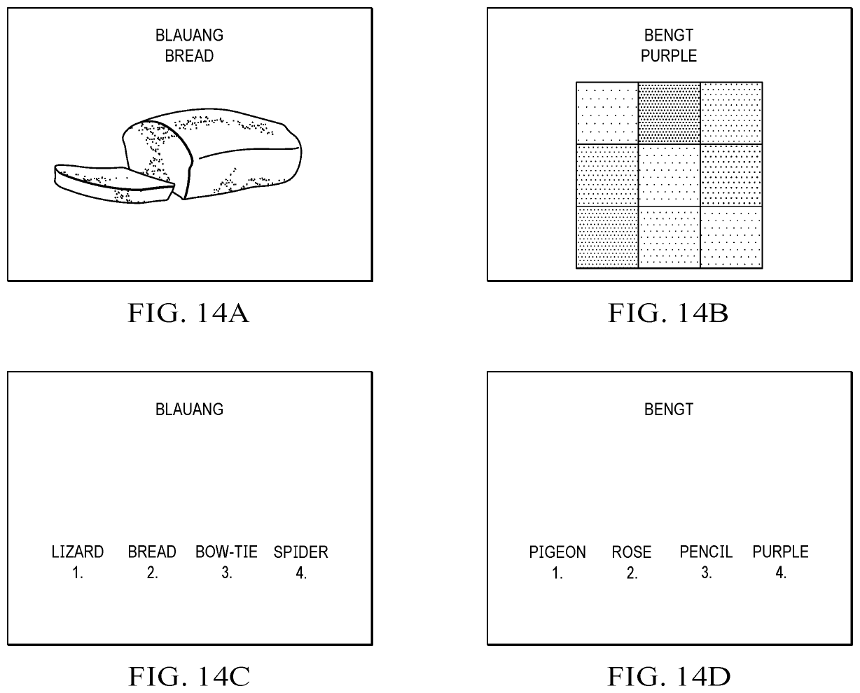

During each exposure block, a total of 25 trials were presented, each consisting of a Palauan noun, its English translation, and an image representing the word's meaning (FIGS. 14A and 14B). Each trial was presented for four seconds and each block contained a randomized set of five words, each presented five times in pseudorandom order so that no word was presented twice in a row. Each exposure block lasted 100 seconds. Following each exposure block, stimulation was turned off and participants completed a short testing block (knowledge check) to evaluate attention during training and familiarity with the words learned in the preceding exposure block. In each knowledge check trial, participants saw a Palauan word and were instructed to pick the correct English translation out of four possible choices (FIGS. 14C and 14D). Each testing block included five trials of each of the most recently learned words for a total of 25 trials presented in a pseudorandomized order. Reaction time and accuracy data were also collected during knowledge check trials. The training session was presented using custom coding in PsychoPy (Peirce et al., 2019).

Participants completed a total of 12 exposure blocks and 12 knowledge check blocks with an optional short break at the halfway point. In total, participants viewed each of the 30 words a total of 10 times. No adverse events occurred during training.

### Palau Free-Recall Translation Assessment

To assess learning and retention, participants completed a free-recall assessment immediately following training (i.e., post-training) and seven days after training (i.e., retention). Participants were instructed to provide the English translation to each of the thirty words learned during training. The assessment was administered through Qualtrics (Qualtrics, Provo, Utah), with the 30 test words presented in a randomized order. There was no time limit on this task. The assessment was scored such that each correct translation earned one point, and each incorrect translation earned zero points. Raw scores out of thirty possible points were converted to percent correct.

### Statistical Analysis Plan

All data were analyzed using custom scripts in MATLAB (MathWorks, Natick, Mass.). A one-way between-groups ANOVA was used to ensure groups were matched on baseline assessments of IQ, reading, memory, and attention (FIG. 10). Descriptive statistics for standard assessment scores are presented as M±SD. A mixed design ANOVA of stimulation group (sham taVNS, 5 Hz taVNS, vs. 25 Hz taVNS) and test time (post-training and retention) was used to evaluate free-recall translation performance. Significant main effects were followed up with post-hoc t-tests. Descriptive statistics for outcome measures are presented as M±SEM. All t-tests were unpaired and two-tailed unless otherwise noted.

Given the significant difference in thresholding current intensities between the two active stimulation groups, we applied partial correlations, controlling for stimulation frequency, to evaluate whether stimulation intensity was significantly correlated with performance outcomes. Additional Pearson's correlations were used to evaluate relationships between baseline verbal learning scores and performance on the free-recall assessments separately for each experimental group. The Bonferroni correction was applied to account for multiple comparisons.

### Results

Higher Frequency taVNS Improves Retention of Trained Words

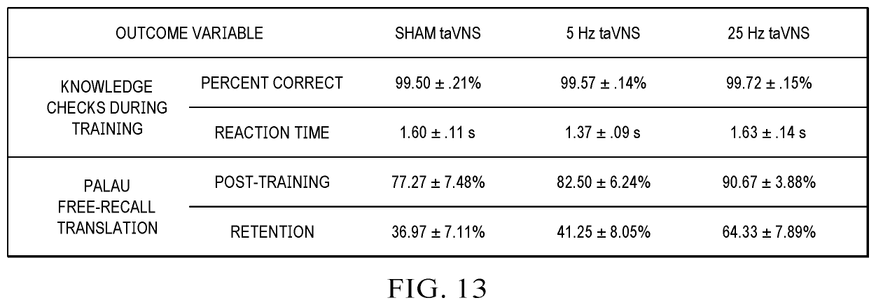

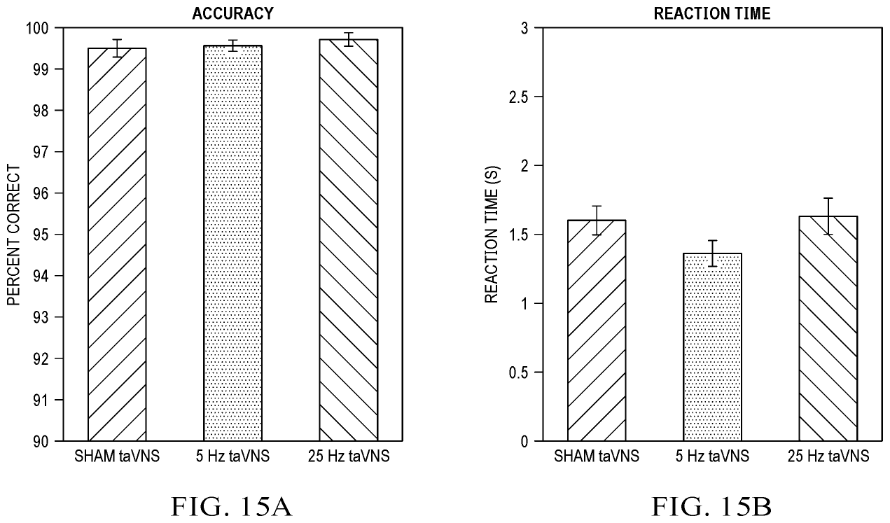

We first analyzed accuracy and reaction time data during knowledge checks to evaluate attention and learning over the course of the training program using one-way ANOVAs (FIG. 13). There was no main effect of group on accuracy during training (F (2, 26)=0.49, p=0.62, η²=0.04; FIG. 15A). There was also no main effect of group on reaction time (F (2, 26)=1.56, p=0.23, η²=0.11; FIG. 15B). Importantly, accuracy performance in all groups was close to the ceiling and confirmed that participants were paying attention and gained familiarity with novel vocabulary words throughout the training session.

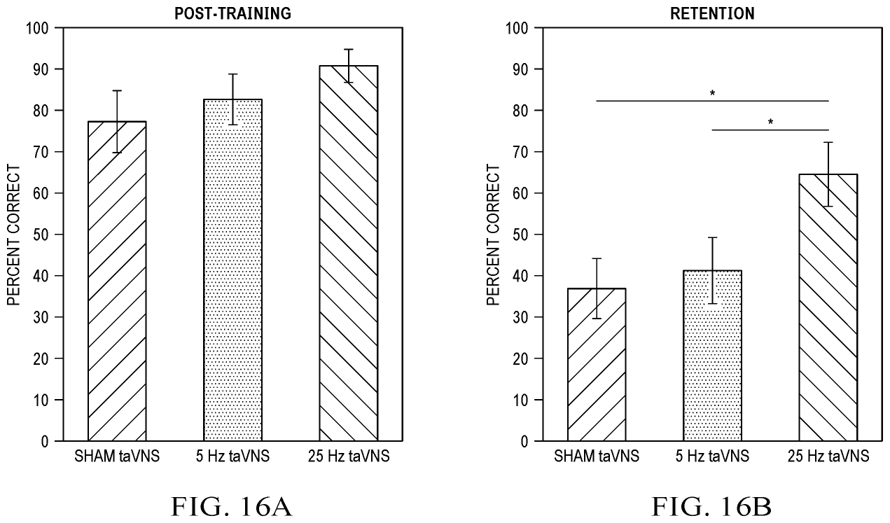

We next analyzed performance on the post-test and retention translation tests. There was a significant main effect of test time (F (1, 26)=117.20, p<0.001) and a marginal main effect of stimulation group (F (2, 26)=3.36, p=0.05). There was no interaction between stimulation group and test time (F (2, 26)=2.16, p=0.14; FIG. 13). Post hoc t-tests revealed there was no effect of taVNS at post-training (ps>0.12; FIG. 16A). However, at retention, the 25 Hz taVNS group outperformed both the sham taVNS group (t (19)=2.72, p=0.01, d=1.19) and the 5 Hz taVNS group (t (16)=2.15, p=0.048, d=1.02; FIG. 16B). There was no significant difference between the sham and 5 Hz taVNS groups (t (17)=0.42, p=0.68).

No Effect of Stimulation Intensity on taVNS Efficacy

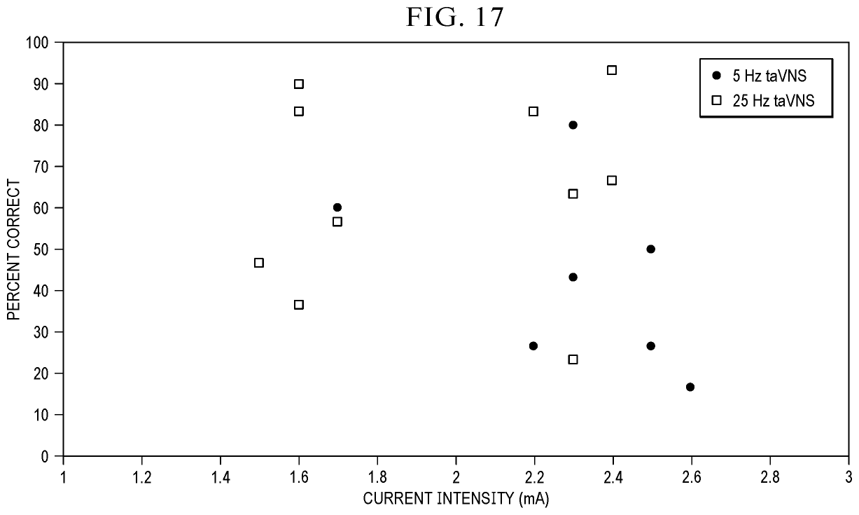

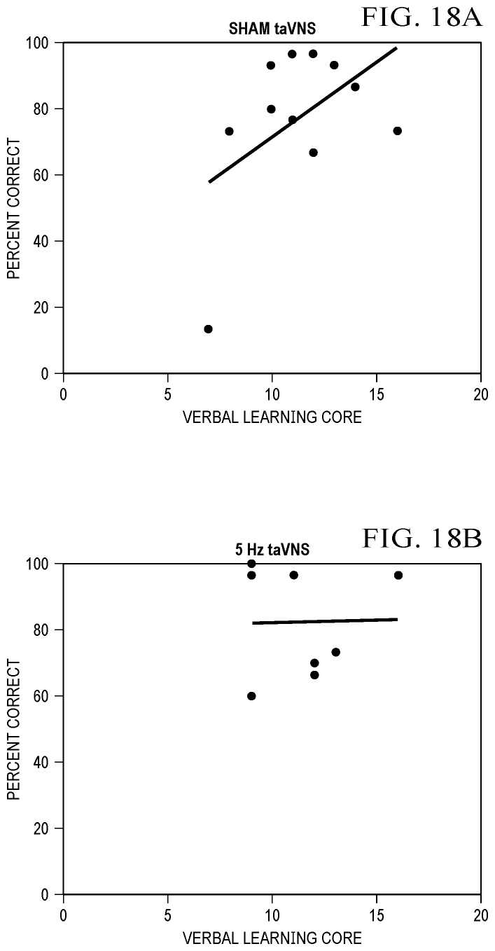

Since there was a significant difference in the thresholding intensity in each stimulation group, we evaluated whether the effect of stimulation frequency on retention performance was driven by differences in current intensity. Partial correlations, controlling for stimulation frequency, were not significant at retention (r=0.27, p=0.17; FIG. 17). These results suggest that stimulation intensity did not impact performance and that stimulation frequency did not influence this finding.

Verbal Memory Ability Predicts Retention Performance in the 25 Hz taVNS Group

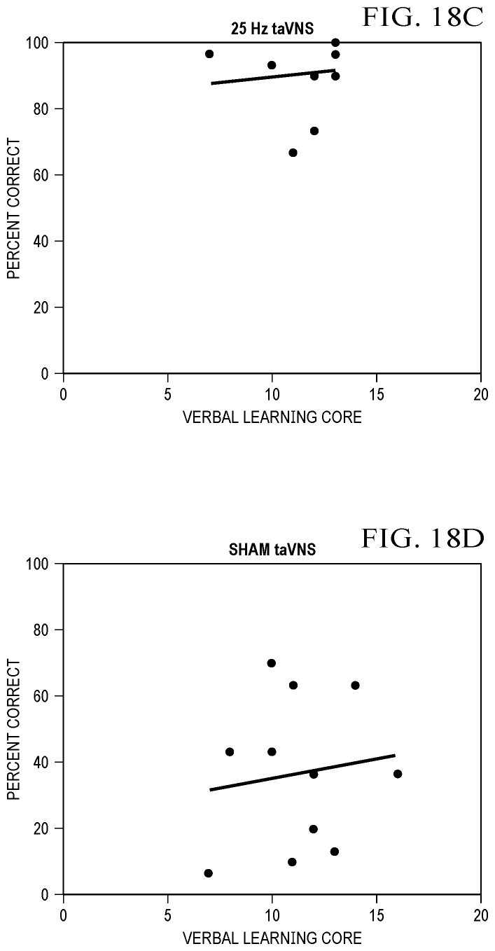

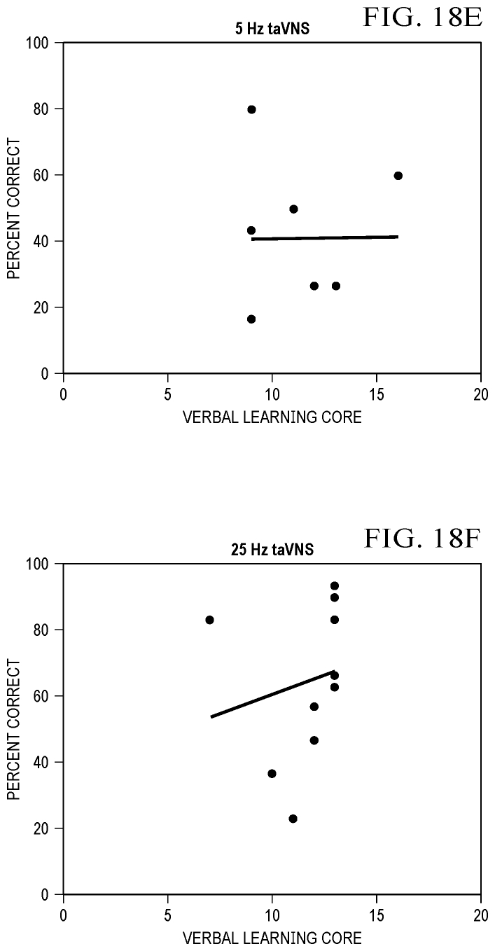

The ability to acquire knowledge of vocabulary words in a novel language requires a variety of baseline skills, including working memory (Atkins & Baddeley, 1998). To evaluate whether verbal working memory skills provided an advantage to participants, we used Pearson's r to quantify the relationships between our measures of learning and each of two Verbal Learning subtests of the WRAML-2 (Core and Recall). During the Core subtest, participants listened to a list of words and immediately repeated as many as they could remember, in any order. This list was read and repeated four times in succession. There were no significant relationships between the standard scores on the Verbal Learning Core and post-training performance within any group (ps>0.13; FIGS. 18A-C). There were also no significant relationships between Verbal Learning Core and retention scores (ps>0.61; FIGS. 18D-F). The lack of any relationship between Verbal Learning Core and outcomes was likely due to fact that this assessment evaluates short-term (10-30 seconds) memory, while language learning requires consolidation, especially for long-term retention.

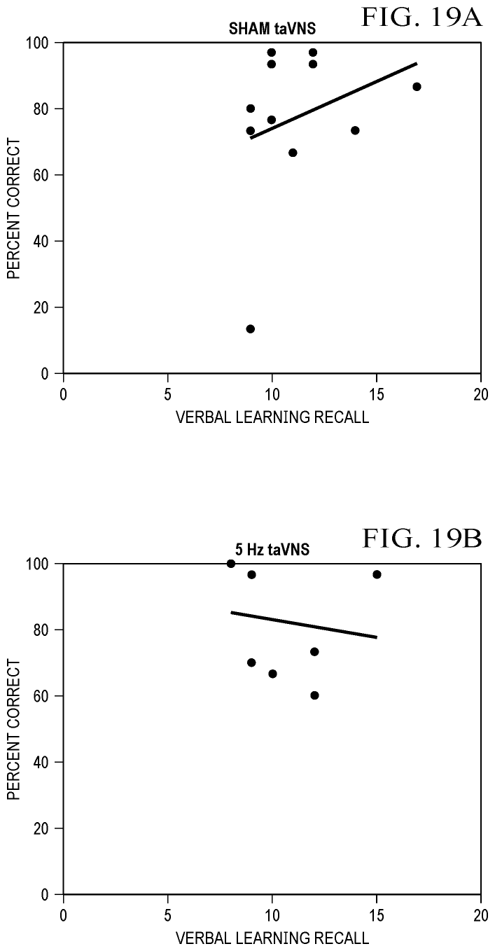

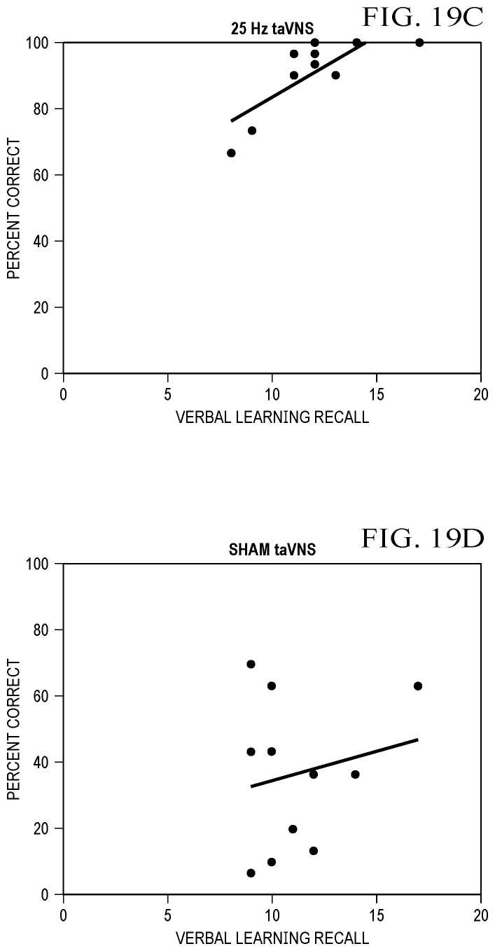

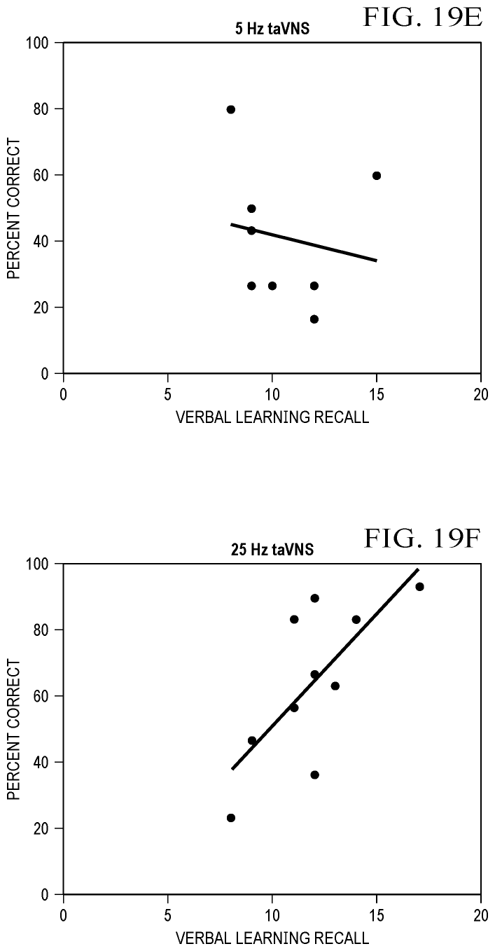

Thus, we next evaluated whether a longer-term verbal working memory task, the Verbal Learning Recall task, was a better predictor of performance. This subtest includes a 10-15 minute delay between initial presentation of a word list and the prompt for free-recall assessment. There were no significant relationships between this measure and post-test performance in either the sham (r=0.30, p=0.37) or 5 Hz (r=−0.17, p=0.70; FIGS. 19A and B) groups. However, there was a significant, positive relationship in the 25 Hz taVNS group (r=0.79, p=0.007; FIG. 19C), such that higher scores on verbal recall correlated with better post-training performance, though this finding did not survive correction. At retention, there was no significant correlation between Verbal Learning Recall and performance in the sham (r=0.20, p=0.56; FIG. 19D) or the 5 Hz taVNS (r=−0.17, p=0.69; FIG. 19E). There was a significant, positive relationship between Verbal Learning Recall and performance within the 25 Hz taVNS group (r=0.72, p=0.018; FIG. 19F), but this finding also did not survive correction.

### Discussion

The aim of the current study was to investigate the effectiveness of taVNS on typically developing young adults' ability to learn and retain vocabulary words in a new language and whether stimulation frequency and/or intensity influenced performance. We observed no effect of taVNS on learning of words immediately after training, but a significant benefit of 25 Hz stimulation on retention of learned words after a seven-day delay period. Importantly, while frequency influenced efficacy, there was no effect of stimulation intensity on performance. These findings demonstrate, for the first time, that taVNS can aid in novel vocabulary retention and that stimulation frequency may be an important parameter to consider when designing taVNS protocols.

taVNS with Learning and Retention

While previous taVNS studies have shown significant effects after training (e.g., Llanos et al., 2020; Redgrave et al., 2018), our results suggest that there is no benefit of taVNS during initial vocabulary acquisition, but significant findings emerged at the seven-day retention. Learning during training was measured by accuracy and reaction time during the 300 knowledge check trials. It is likely that the null effect of taVNS on acquisition during training was due to the close temporal proximity between exposure to the new words and the knowledge checks pertaining to those words. Participants in all groups performed at ceiling levels on these knowledge checks, leaving little room to detect any benefits of taVNS. Since the acquisition task was designed to mimic well-known language learning approaches, such as those used by DuoLingo and Rosetta Stone, it is possible that the training approach was maximally effective.

To investigate potential effects of taVNS during training, and in one embodiment, the present disclosure contemplates utilizing a training program lasting multiple days, utilizing a drop-out training method, or restructuring the training program to increase difficulty. A drop-out training method, as used in Swahili-English learning, only has participants restudy and tested on words that are not learned over the course of the task, and words not correctly remembered would be maintained in the task (Pasqualotto, Kobanay, & Proulx, 2015). This procedure for knowledge checks may detect potential taVNS benefits more accurately, since words not learned well stay within the training program. In such an experiment, data could be analyzed as number of trials it takes to learn the new word consistently. Another potential approach could be to redesign the knowledge checks in a more difficult format (e.g., written free-recall, matching, or verbal recall), further probe the potential effects of taVNS during initial learning. Using these methods makes it less likely that participants will exhibit performance at ceiling during training. Alternatively, to further mimic previously used computer-training programs, such as DuoLingo, knowledge checks could be administered in a matching format. For example, multiple novel words or pictures can be listed in one column with corresponding translations in another column, and participants would have to correctly match novel word or picture to English translation. An extra layer of difficulty could be added if words presented were a combination of words from the current and previous training blocks.

A second suggestion to probe the potential effects of taVNS during training would involve restructuring the training program format. In the current study, participants saw five repetitions of five words and then completed knowledge checks on the words they had just seen. Another approach to address this could be that participants see ten words per block to increase the information they need to remember for subsequent knowledge check trials. Given the previous research suggesting the importance of memory skills in vocabulary learning (e.g., Wojcik, 2013), increasing the number of words to be remembered may help detect potential benefits of taVNS. Despite a null finding during training, the ceiling effect does demonstrate the feasibility of the training program, since all participants in all groups gained familiarity with novel word associations in a single training session.

Immediately after training and seven days after training, participants completed a free-recall test, which is more difficult than a multiple-choice knowledge check, of trained stimuli by providing English translations to the Palauan words. We observed no effect of taVNS on the free-recall assessment that occurred immediately after training. This finding poses the question about the effect of taVNS on overall learning after training (see Colzato & Beste, 2020, for a review). For example, a cross-sectional study comparing young adults to older adults found no benefit of taVNS to the cymba conchae on verbal memory learning in either age group, both during the study (analogous to knowledge checks in the current study) and after the study (analogous to the post-training free-recall test in the current study; Mertens et al., 2020). In another study, taVNS applied to the posterior tragus led to higher performance in association memory in older adults ten minutes after a single session (Jacobs et al., 2015). The delay between training and testing supports the hypothesis that vagus nerve stimulation strengthens memory for learned associations. As a third example, we did previously find benefits of 5 Hz taVNS, but that was after five days of training (Thakkar et al., 2020), but data from that study were not scored during training (i.e., knowledge checks), so it is uncertain if taVNS was beneficial during training (knowledge checks) or just at post-training. Collectively, these studies may suggest that taVNS may have stronger effects later, rather than during the course of training.

taVNS Efficacy is Impacted by Stimulation Frequency but not Intensity

While a number of prior taVNS studies have demonstrated significant effects by utilizing a range of stimulation frequencies, no study to date has systematically compared multiple stimulation frequencies within the same paradigm. The current study is the first to show that frequency of stimulation may influence its efficacy on language learning. We directly compared multiple frequencies, which were chosen based on prior successes in reading and language at both 5 Hz (Thakkar et al., 2020) and 25 Hz (Llanos et al., 2020). In the current study, we observed a significant benefit of 25 Hz stimulation, but not 5 Hz stimulation, on vocabulary recall performance seven days after training. Although this result provides support for 25 Hz as an effective stimulation frequency, it is important to emphasize that our findings do not suggest that 25 Hz should be used in all taVNS studies. Similarly, while the current study did not show a benefit at 5 Hz, we do not suggest that researchers abandon this frequency, as our prior work did demonstrate significant benefits of 5 Hz stimulation on automaticity (i.e., rapid naming) and decoding (i.e., timed pseudo word reading), in typically developing young adults learning novel letter-sound relationships in Hebrew (Thakkar et al., 2020).

There are a number of possible reasons for the discrepancy in the 5 Hz taVNS effect across our prior study and the current study. First, the stimulation location differed across studies, as our prior study stimulated the cymba concha region of the left ear (Thakkar et al., 2020) and the current study stimulated the posterior tragus of the left ear. This change was due to availability of stimulation devices rather than a methodological choice and suggests that additional research is needed to determine whether stimulation to the posterior tragus or the cymba concha impacts efficacy. Second, the two studies differ in the number of pairings per stimulus, which refers to the number of times a stimulus is presented or taught when paired with stimulation. It is possible that there is a threshold of minimum number of pairings necessary for taVNS to be rendered effective, and previous protocols have varied in the number of pairings used. Our prior study taught 16 consonant vowel-combinations with approximately 215 pairings per stimulus, while the current study taught 30 picture-word combinations with ten pairings per stimulus. While cVNS and taVNS are both well-supported through multiple studies, many pairings between stimulus and stimulation are needed to drive behavioral benefits and neural plasticity. Motor function was recovered in post-stroke patients after receiving 30-50 repetitions of each motor movement paired with taVNS (Redgrave et al., 2018). A third possibility is the type of content being learned. In our letter-sound learning study, we utilized an audio-visual stimulus where participants mapped a visual grapheme to a corresponding sound, whereas the current study taught vocabulary words where participants mapped printed words to corresponding pictures with no auditory stimulus component. Based on the type of content being learned, it is possible that information was processed or modulated through different neural circuits. In another embodiment, the present disclosure can include and be advantageous in understanding the impacts of stimulation location (posterior tragus vs. cymba concha), training length (number of pairings and number of sessions), and frequency (low, moderate, vs. high) interact, all of which can be critical for designing optimal protocols utilizing this technology.

Another parameter of interest evaluated in this study is the relationship between current intensity and performance in each of the active taVNS groups. In the current study, we found no effect of current intensity on learning or retention. Previous research using the invasive cVNS in a rodent model compared various stimulation frequencies and found that moderate intensities of stimulation led to more neural reorganization in auditory cortex than did higher intensities of stimulation (Borland et al., 2016), and these results were confirmed in similar studies of motor cortex (Morrison et al., 2020; Morrison et al., 2021). This influence of intensity was also observed in humans, where cVNS at moderate intensity yielded stronger effects than at higher intensity during a verbal memory task (Clark et al., 1999). These previous cVNS studies suggest that moderate current intensities are most effective (see Van Leusden, Sellaro, & Colzato, 2015, for a review), but little is known about whether intensity influences efficacy in taVNS. One prior study investigated the effects of subthreshold stimulation on categorization of speech sound categories in Mandarin. They reported that subthreshold taVNS selectively enhanced learning on certain categories (Llanos et al., 2020). This finding, in tandem with our taVNS effect using suprathreshold intensities, suggests that taVNS efficacy may rely more on the matching of frequency to the task at hand rather than matching stimulation intensity along an inverted-U function. The present disclosure can also include variations in the frequency and intensity parameters across a range of tasks (Groves & Brown, 2005).

### Baseline Working Memory and Performance

Previous research has established the importance of memory skills in vocabulary learning and retention. In order to successfully acquire novel vocabulary words, a learner must associate a word with a form of meaning. Children are able to do this easily with mere exposure, but regardless of exposure or experience, we must remember these associations for the future (Wojcik, 2013). Previous work suggests that there is a definite connection between verbal short-term memory and novel word learning (Gupta & MacWhinney, 1997; Jarrold, Thorn, & Stephens, 2009; McGregor et al., 2017). Thus, we also evaluated the relationship between a subset of measures in the WRAML-2 (Sheslow & Adams, 2009) and performance on the translation tests, as verbal working memory has been linked to novel word learning in children (Hansson et al., 2004). We observed a significant relationship between the Verbal Learning Recall measure and retention performance, specific to the 25 Hz taVNS group. This could be attributed to a compounding effect of verbal recall and stimulation, since there was a benefit of high frequency stimulation on performance at retention. The memory tasks in the present study test how many words participants can name after hearing them in serial order for four trials (Verbal Learning Core) and after a brief delay (Verbal Learning Recall). Thus, it can be speculated that those with higher vocabulary memory at baseline can more easily acquire novel vocabulary words. Biologically, we infer that taVNS takes advantage of that relationship to amplify long-term retention, given the results obtained in the current study. This finding may be corroborated by previous imaging work that found higher performance on verbal learning tasks is associated with greater activation of key brain regions, which also overlaps with the language network (Heinze et al., 2006). Importantly, there are other popular verbal fluency measures, such as the letter (Newcombe, 1969) and category fluency (Benton, 1968) tasks, that are widely used in assessment. Tests like these test participants' abilities to name as many items (vocabulary terms) specific to a letter (F, A, S) or category (animals, food) as they can in a given unit of time. These tests can broadly assess vocabulary size, since it gives participants the ability to draw from their entire vocabulary. Based on previous studies (Kaushanskaya & Marian, 2009), it could be predicted that those with greater vocabulary sizes in English, as measured by number of words listed in verbal fluency tasks, can learn and retain more words in a novel language. The present disclosure can include and be advantageous in understanding how verbal learning tasks (e.g., Verbal Learning Core, Verbal Learning Recall, Semantic Fluency, and Letter Fluency) interact with stimulation to aid in vocabulary learning. The present disclosure can include and be advantageous in understanding how taVNS, and potentially other neuromodulation techniques, take advantage of prior skills and neural circuits to increase lengthened retention of learned vocabulary words.

Comparison with Other Neuromodulation Techniques

While the current study yielded significant results, not all individuals may benefit from taVNS intervention. There may be certain contraindications of treatment that prevent an individual from being able to use this technique (e.g., disrupted neurotransmitter systems important in VNS, injury to the left ear). Thus, it is important to compare the efficacy of taVNS with other techniques important in vocabulary learning. Collectively, non-invasive brain stimulation techniques have been shown to treat a variety of conditions and enhance learning and memory. For example, transcranial random noise stimulation (tRNS), enhanced acquisition of Swahili words in young adults when stimulation was applied to the posterior parietal cortex (Pasqualotto, Kobanbay, & Proulx, 2015). Another example of a non-invasive neuromodulation technique is transcranial direct current stimulation (tDCS) which operates through a mechanism of priming neurons for learning through cortical excitability (Medeiros et al., 2012). When using tDCS, anodal stimulation to a specific region of interest (ROI) is used to increase excitability of underlying neurons, and cathodal stimulation to a specific ROI can decrease excitability. Prior studies using tDCS have found that anodal stimulation to the temporo-parietal cortex led to faster reaction times in retrieval after a word-picture training program of learning Finnish farming equipment (Perceval et al., 2017). tDCS over Wernicke's area led to enhanced vocabulary learning at the end of training blocks and with overall learning (Floel et al., 2008). Anodal tDCS to the left inferior frontal gyms (IFG) also yielded higher accuracy scores in a verb learning task in later blocks of training in healthy young adults (Fiori et al., 2018).

taVNS operates through a mechanism of releasing key neurotransmitters implicated in learning memory by pairing stimulation with a particular external stimulus. taVNS devices are smaller and more easily accessible compared to other non-invasive neuromodulation techniques, so pairing taVNS devices can serve as a feasible addition to computer training programs in language learning. In another embodiment, the present disclosure can include various noninvasive neuromodulation techniques, such as taVNS, tRNS, and transcranial direct current stimulation (tDCS), since each operates through a unique mechanism.

### Limitations

There are four main limitations to the current study. First, our sample size is smaller than we had planned for and anticipated. Unfortunately, our recruitment of participants was cut short by the COVID-19 pandemic, which created conditions that made in-person data collection unsafe. Thus, we have plans to replicate and extend these findings in a larger, fully powered study. An a priori power analysis (f=0.25, a=0.05, power=0.90, groups=3, measurements=2, correlation among repeated measures=0.5) suggested that a total sample size of 54 participants would yield a significant effect and moderate effect size. Using the strongest effect at retention, a post-hoc power analysis (d=1.19, a=0.05, nₛₕₐₘ=11, n_{25 Hz}=10) suggested a power of 0.73, meaning that it is underpowered (Faul et al., 2007).

A second limitation of the current study was that we only included typically developing young adults that were all college undergraduates from high socioeconomic backgrounds. As such, our findings may not represent the general population, and that poses a limitation since certain demographic variables, such as socioeconomic status and parenting, can influence language development (Fernald, Marchman, & Weisleder, 2013; Perkins, Finegood, & Swain, 2013).

A third limitation was a restriction on the type of words taught during training. We trained thirty concrete nouns in one training session. Concrete nouns were used so that a visual image could be provided with the translation. Prior research has also shown that concrete nouns are more easily remembered than abstract nouns (e.g., Fliessback et al., 2006; Hamilton & Raj aram, 2001). This poses a limitation since vocabulary in language learning must also involve the learning of abstract nouns (e.g., loyalty, love, excitement), and not just concrete nouns (e.g., bread, spoon, rose), and it is currently unknown whether taVNS will be effective in enhancing learning for abstract vocabulary words. Additionally, proficiency in a language also requires knowledge of verbs, adjectives, and grammar skills, which were not taught in the current study.

A fourth limitation was the lack of neural recordings taken in the current study. Neuromodulation techniques, such as cVNS and taVNS, are designed to induce neural plasticity. There is well-documented evidence in cVNS leading to neural plasticity in the sensory and motor cortex (Engineer et al., 2001; Borland et al., 2016). While taVNS studies (e.g., Llanos et al., 2020) saw behavioral benefits, there was not adequate neural plasticity. Thus, the lack of neural recordings in the present study prevents us from drawing any conclusions about high frequency taVNS inducing neural plasticity that align with the behavioral benefits seen.

### EXAMPLE #3

### Introduction

Fluent reading is a necessary skill in the developed world but in spite of adequate education and intelligence, up to 20% of children fail to acquire adequate reading comprehension skills. Difficulty in reading comprehension causes significant hardship not only with respect to self-esteem, but also to academic and vocational outcomes. For example, students are required to read and comprehend material from textbooks and lecture slides, utilize the printed word for studying, and then for reading and completing exams. While reading and comprehension skills are essential, acquiring these skills takes years of practice and instruction. Despite practice and instruction, approximately 10% of the population fails to acquire adequate reading and comprehension skills (Pennington & Bishop, 2009). While a number of reading comprehension interventions exist, they are time consuming and not effective for all users, especially those targeted at adults. Thus, the aim of the current study was to investigate a novel approach to improve reading comprehension scores in young adults.

A number of widely-used interventions aim to improve oral reading skills and reading comprehension skills. For example, in adult ESL learners, a vocabulary-based training improved literacy, but not comprehension (Oliver & Young, 2016). As a second example, researchers adapted reading intervention programs that were effective in children (i.e., Corrective Reading, RAVE-O, and Guided Repeated Reading) and found that, in adults, these interventions can improve multiple skills, including comprehension, after 10-18 weeks of intervention (Sabatini et al., 2011). As well, their data suggested that no intervention program was more beneficial than another, but behavioral intervention, broadly, aided in these skills. While these interventions show some promise, they exhibit three main limitations: 1) they take many weeks of intervention, 2) they do not fully address executive functions implicated in reading comprehension, such as working memory, and 3) they are not effective in all cases. A method to decrease intervention time would be incredibly valuable for individuals who need to improve their performance at school or at work.

Neuromodulation is a popular approach to enhance cortical connections and drive neural plasticity. One such method is cervical vagus nerve stimulation (cVNS), as stimulation of the vagus nerve releases norepinephrine and acetylcholine, neurotransmitters implicated in learning and memory (Picciotto, Higley, & Mineur, 2012; Tully & Volshakov, 2010). Importantly, key neurotransmitter systems must be intact to ensure effectiveness of cVNS (Hulsey et al., 2019). In the sensory domain, cVNS paired with an external stimulus, such as a tone, leads to significant and long-lasting neural plasticity in the rodent primary auditory cortex (Engineer et al., 2001). cVNS paired with training has also improved motor function recovery in stroke-induced rats and driven neural plasticity in motor cortex (Khodaparast et al., 2013; Porter et al., 2012). In humans, cVNS leads to higher rates of recognition memory of highlighted words in a passage (Clark et al., 1999), decreased errors in a delayed recall task (Sun et al., 2017), and better performance on a verbal fluency task (Sackeim et al., 2001), when compared to sham cVNS. To date, cVNS has been FDA approved for individuals with treatment resistant epilepsy and depression, and it is in active clinical trials for stroke and tinnitus. However, cVNS implantation requires an expensive and invasive procedure, which makes it an impractical intervention for reading skills.

Transcutaneous auricular vagus nerve stimulation (taVNS) activates similar deep-brain structures as cVNS (e.g., locus coeruleus), without the need for an invasive, expensive surgery, by applying low-level electrical stimulation to the left outer ear (Badran et al., 2018; Frangos, Ellrich, & Komisaruk, 2015; Yakunina, Kim, & Nam, 2016). Growing evidence supports the hypothesis that taVNS drives similar neural plasticity as the more invasive cVNS. For example, taVNS paired with physical therapy increases post-stroke motor function recovery performance (Redgrave et al., 2018) and alleviates symptoms of tinnitus (Yakunina, Kim, & Nam, 2018). In the language and reading domains, taVNS improves learning of novel letter-sound pairings (Thakkar et al., 2020) and novel Mandarin speech sound categories (Llanos et al., 2020). This approach also increases performance on a face-name association task in healthy older adults (Jacobs et al., 2015), suggesting its effectiveness in higher cognitive domains. Given the previous evidence that taVNS increases performance in learning and reading tasks, we evaluated whether taVNS is capable of improving reading comprehension performance, a skill requiring both reading and memory, in typically developing young adults.

### Methods

### Participants

Fifty-five young adults were screened for eligibility from an online participant pool at Texas Christian University in Fort Worth, Tex. All potential participants completed a short online background survey covering personal history of reading and motor development, diagnoses, medications, and family history. Participants then completed several baseline assessments including: a non-verbal IQ measure (the matrices subtest of the KBIT-2; Kaufman & Kaufman, 2004), timed single-word reading (the Sight Word Efficiency and Phonemic Decoding Efficiency from the TOWRE-2; Torgesen, Wagner, & Rashotte, 2012) and untimed single-word reading (Word Identification and Word Attack from the WRMT-3; Woodcock, 2011). To qualify as a typical reader, participants needed to (a) be a native English speaker, (b) be between the ages of 18-35, (c) achieve a standard nonverbal IQ score of 85 or higher, (d) achieve standard reading scores of 90 or higher on all four measures, (e) report no neurological injuries, disorders, diagnoses, or medications, and (f) have no medical implants. In addition to inclusion testing, participants also completed Passage Comprehension and Oral Fluency (WRMT-3; Woodcock, 2011), Rapid Digit and Letter Naming (CTOPP-2; Wagner, Torgesen, & Rashotte, 2013), and the Design Memory Core, Verbal Learning Core, Number-Letter, Design Memory Recognition, and Verbal Learning Recall subtests (WRAML-2; Sheslow & Adams, 2009).

Of the participants who completed screening, 11 were excluded for low reading scores, five for low IQ, six for exclusionary medications or diagnoses, three for safety concerns related to the placement of the taVNS device, two for scheduling conflicts, and four withdrew partway through the study. Thus, the final sample included 24 typically developing young adults (age: 19.84±0.40 years; 7 males and 17 females). Only typically developing young adults to ensure participants had intact neurotransmitter systems to investigate the effectiveness of taVNS on reading comprehension. Eligible participants were randomized into a sham taVNS (n=12) or active taVNS (n=12) group.

While we originally planned to recruit a larger sample size in the study, recruitment was terminated due to safety concerns of in-person research and potential confounds as a result of the COVID-19 pandemic. To maximize our statistical power in these circumstances, each experimental group consisted of twenty participants that completed the study using a Parasym taVNS device (https://www.parasym.co/index.html) and four additional participants that completed the study using the TENS7000 transcutaneous electrical nerve stimulation (TENS) device (https://tens7000.com/). To ensure that the difference in stimulation device did not impact the results, we conducted a confirmatory analysis in the sample trained using the Parasym device. All participants were compensated with course credit, and the study was approved by the Texas Christian University Institutional Review Board. All participants provided written informed consent prior to enrollment.

taVNS Device, Settings, and Procedures

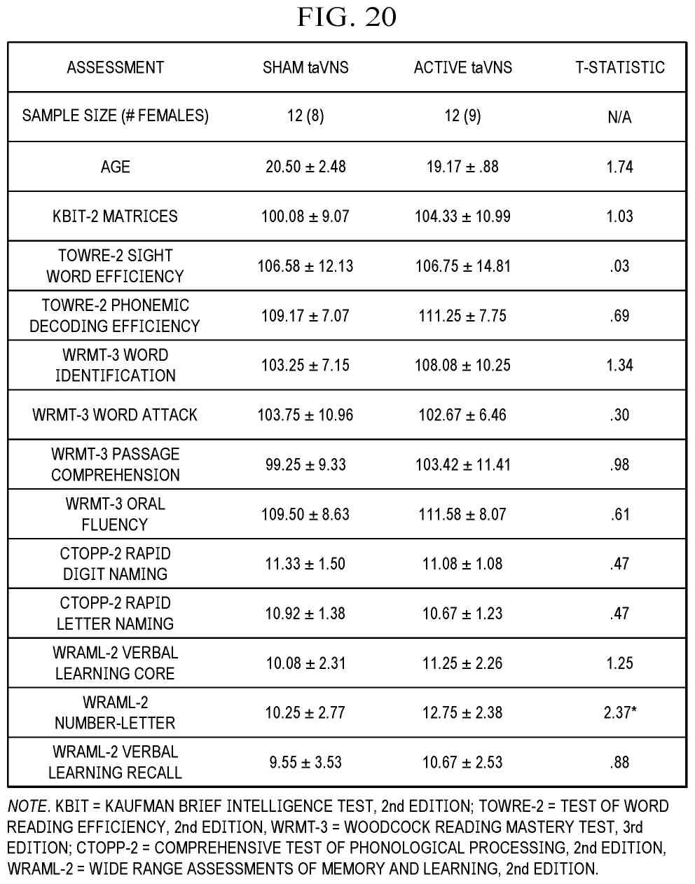

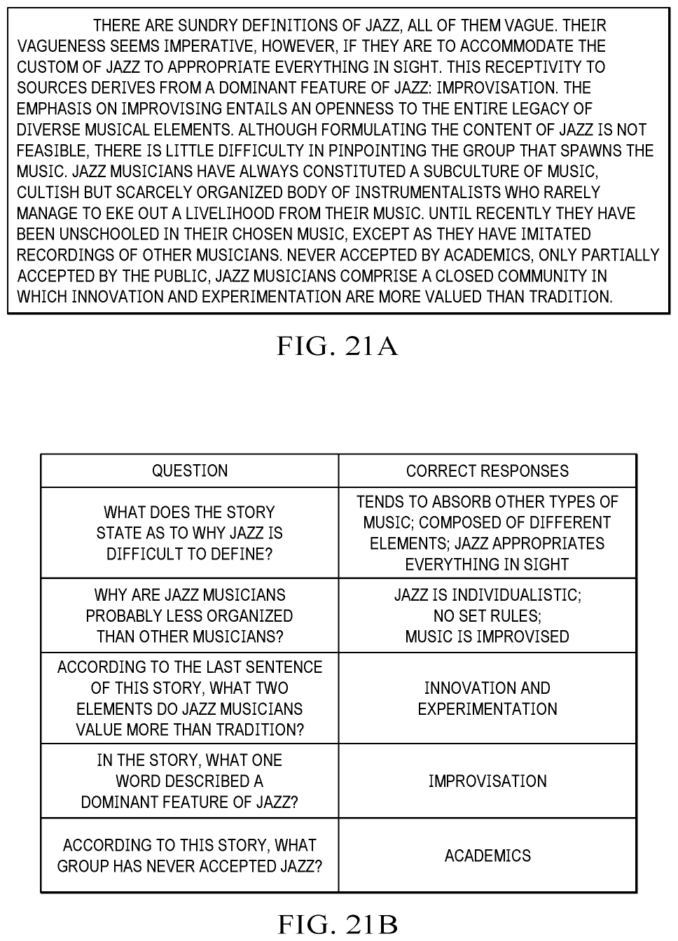

Most participants (nsham=10, nactive=10) received taVNS from the ParaSym device, which utilizes a one-quarter inch diameter gold-plated copper electrode. For a subset of participants (nsham=2, nactive=2), taVNS was administered using the TENS7000 device. This device utilized an earpiece linked to an electrode with a separate grounding pad placed behind the ear. Regardless of device, the stimulating electrode was positioned at the posterior tragus of the left ear to stimulate the auricular branch of the vagus (Badran et al., 2018; Yakunina, Kim, & Nam, 2016). Current was delivered with identical parameters across devices: as square, biphasic pulses with a 200 μs pulse width, and 5 Hz frequency (Thakkar et al., 2020). During the testing session, stimulation onset and offset were controlled manually by a trained researcher to ensure that stimulation was delivered only during active reading. Stimulation was only delivered during oral reading to model previous research suggesting that VNS efficacy relies heavily on pairing stimulation with the external stimulus, such as a tone or passage being read (Engineer et al., 2011). Custom stimulation intensity was determined for each participant during a short thresholding procedure (FIG. 20). A trained researcher acquired two measurements at each participant's absolute minimum threshold and two measurements at the upper level of comfort, prior to the onset of pain (Thakkar et al., 2020; Yakunina, Kim, & Nam, 2016). The average current threshold was (2.03±0.10 mA), with the sham group exhibiting marginally higher thresholds (2.23±0.11 mA) than the active taVNS group (1.84±0.16 mA; unpaired two-tailed t-test, t (22)=2.69, p=0.057, d=0.83).

### Reading Comprehension Measure: Administration and Data Coding

To evaluate the effect of taVNS on reading comprehension, participants read passages from both forms of the GORT-5 (Wiederholt & Bryant, 2012) in a counterbalanced order. For each form, passage 6 was administered as a practice passage, and passages 11-16 were administered as test passages. Passages were presented in white font on black background using custom code in PsychoPy (Peirce et al., 2019). For each passage, participants read the text out loud at their normal pace and pressed a button when finished, which removed the passage from the screen. Stimulation was manually controlled by an experienced researcher from behind a barrier and was turned on at the initiation of each passage and turned off as soon as the participant finished reading. For those in the sham condition, the device remained off throughout the session without the participants' knowledge. To quantify any effect of taVNS on reading mechanics, we calculated average reading errors per passage (Costanzo et al., 2013) and average reading rate per passage (O'Brien, Mansfield, & Legge, 2005; O'Brien & Wallot, 2016). Reading errors were calculated as the total number of deviations from print per passage (e.g., mispronounced words, added words, omission of words, and changes in the order of words). Reading rate was calculated as the number of words read per minute (wpm) per passage. As there was no effect of test form on overall reading comprehension performance (paired two-tailed t-test: t (23)=0.20, p=0.84), both forms were combined for subsequent analyses.

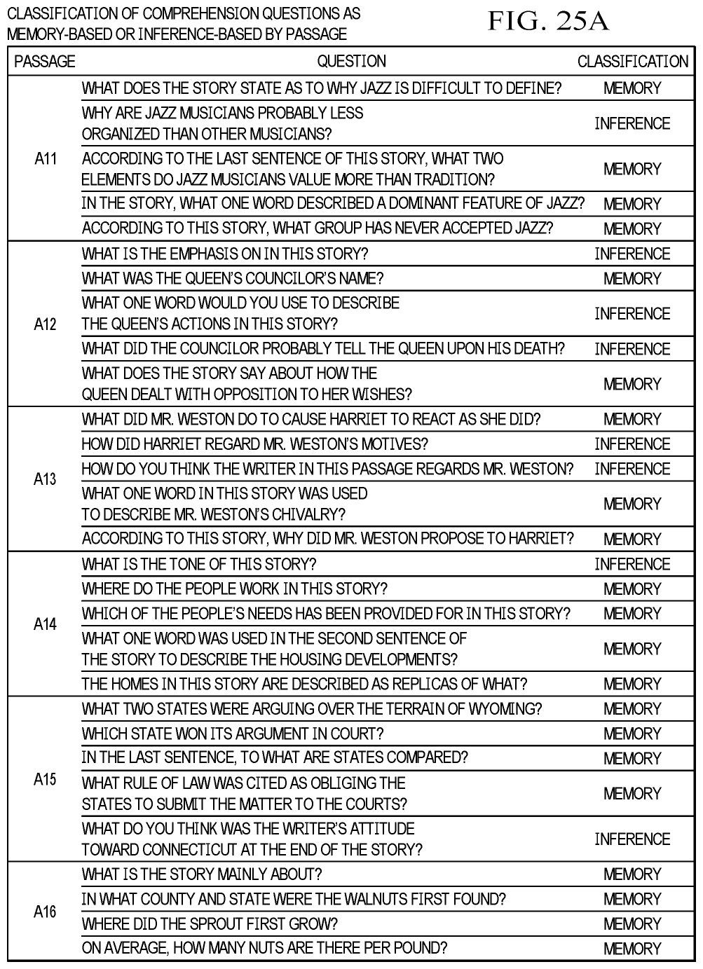

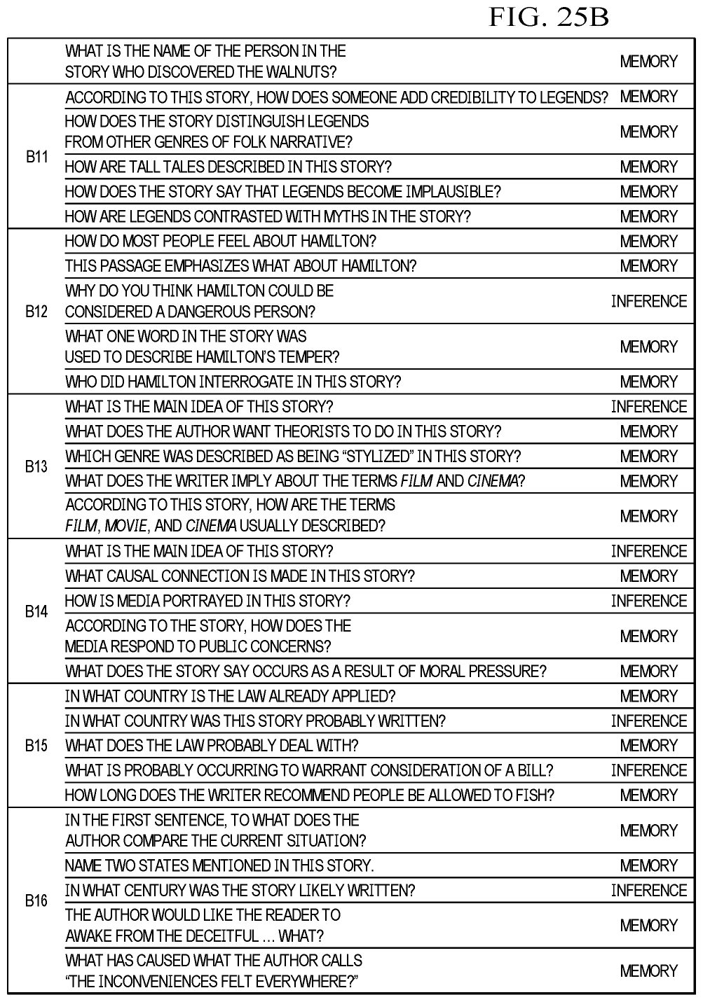

Immediately after reading each passage, a researcher asked the accompanying five comprehension questions as provided in the GORT-5 (Wiederholt & Bryant, 2012). To score the reading comprehension questions, correct answers were awarded one point, and incorrect answers were given zero points, with no partial credit given. Raw scores out of 60 possible questions were converted to a percent correct for further analysis. Two researchers independently scored participant responses for accuracy, and discrepancies were resolved by coming to a consensus. To identify comprehension questions that relied on either working memory or higher cognitive processing, two experienced researchers (authors VT and AD) classified each comprehension question as memory-based (i.e., the answer being explicitly stated in the passage, such as recalling the name of a character named in the passage) or inference-based (i.e., a reader must have an understanding beyond what is explicitly stated in the passage, such as inferring the attitude of the writer of the passage). There were minimal discrepancies, which were resolved by author TMC. In total, 45 questions were classified as memory-based (75% of total questions) and 15 questions as inference-based (25% of total questions). Classification of each individual question is provided in FIG. 25.

### Data Analysis and Statistics

Two-tailed, independent-samples t-tests were used to compare the sham and active taVNS groups on standard English assessments and evaluate any differences in baseline reading abilities. Descriptive statistics are presented as mean±SD (FIG. 20). To investigate the effect of taVNS on reading, we used two-tailed, independent-samples t-tests on each of the key dependent variables, unless otherwise noted. All descriptive statistics for outcome measures are reported as the mean±SEM. Pearson's r was used to determine if variability in individual current intensity was related to comprehension performance in the active taVNS group. Finally, we used Pearson's r to quantify the relationships between two verbal learning measures and comprehension performance. To correct for multiple comparisons, the Bonferroni correction was applied.

### Results

### Standard English Assessments

The sham taVNS and active taVNS groups did not differ on most standard English assessments administered in the initial session (ps>0.19). However, the two groups did differ on the Number-Letter working memory task such that the active taVNS group (12.75±2.38) exhibited higher scores than the sham taVNS (10.25±2.77) group (two-tailed t-test: t (22)=2.37, p=0.03; FIG. 20).

taVNS Improves Performance on Memory-Based Comprehension Questions

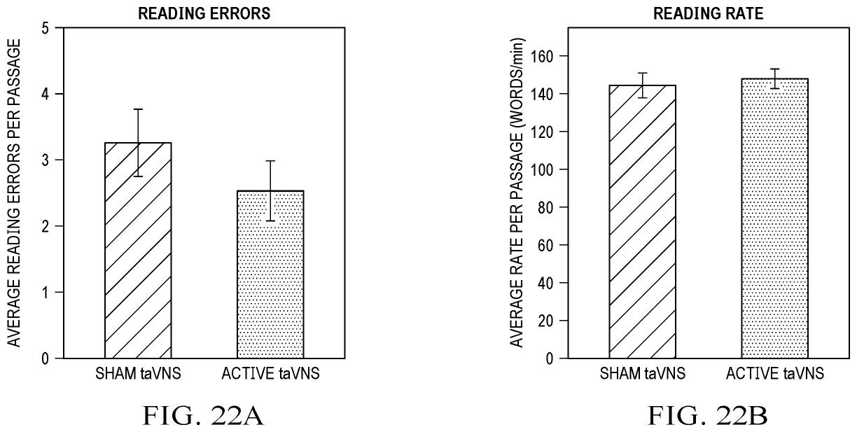

We first evaluated whether taVNS improves reading accuracy or reading rate, as these reading metrics show improvement after repetitive TMS (Costanzo et al., 2013) and may also have benefitted from our stimulation protocol. In terms of accuracy, there was no group difference between sham (3.26±0.51 errors per passage) and active taVNS (2.53±0.46 errors per passage; t (22)=1.12, p=0.28; FIG. 22A). In terms of average reading rate (wpm), there was also no difference between sham (144.38±6.78 wpm) and active taVNS (147.73±4.97 wpm; t (22)=0.42, p=0.68; FIG. 22B). These results suggest that unlike other forms of non-invasive stimulation, taVNS does not influence the mechanics of reading within the session.

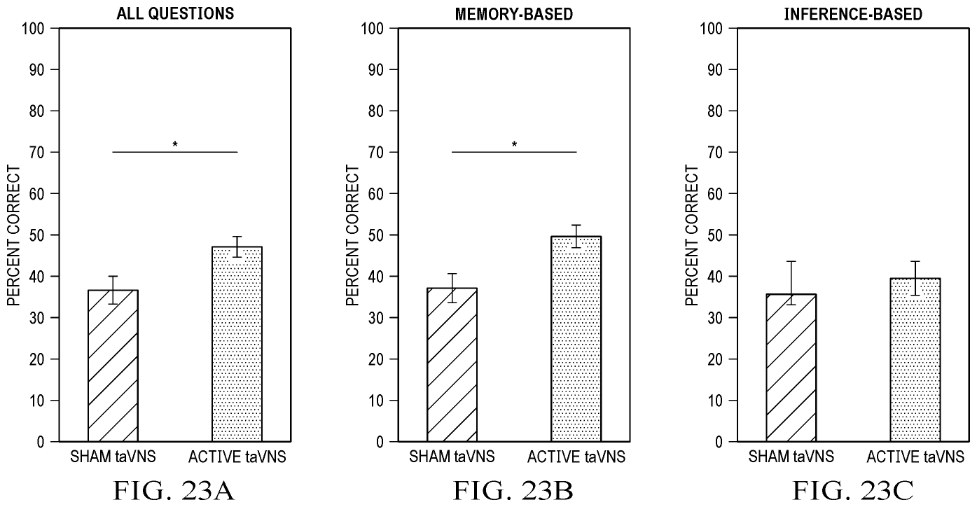

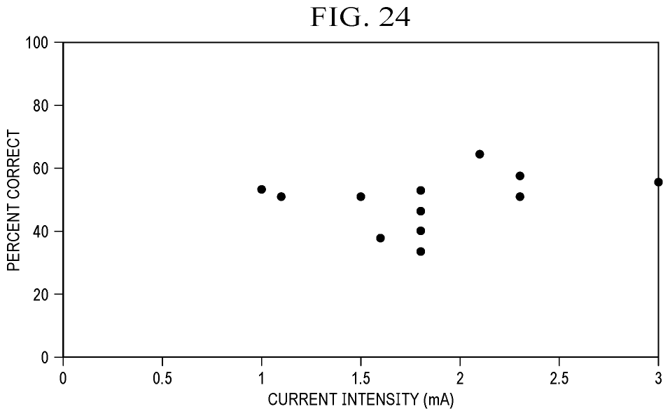

We next evaluated the effect of taVNS on comprehension. There was a significant effect of stimulation across all comprehension questions (t (22)=2.59, p=0.017, d=1.06; FIG. 23A) such that taVNS improved performance compared to sham. To determine whether this effect was driven by performance on a specific question type, we evaluated whether taVNS improved performance on the subsets of working memory and inference questions separately. There was a significant group difference on memory-based questions (t (22)=3.00, p=0.007, d=1.23) such that the active taVNS group (49.63±3.47%) significantly outperformed the sham taVNS group (37.04±3.47%; FIG. 23B). This result survived correction for multiple comparisons. There was no difference between the sham taVNS (35.55±5.24%) and active taVNS group (39.44±4.06%) on the inference-based questions (t (22)=0.61, p=0.54; FIG. 23C).

Since each participant received a custom current level, we evaluated the relationship between current intensity and memory-based comprehension performance in the active taVNS group using Pearson's r. Due to subtle differences in current output across devices, we utilized only the participants that used the Parasym device for this analysis (n=10). There was no significant relationship between current intensity and percent correct (r=0.33, p=0.35; FIG. 24), demonstrating that current intensity did not influence taVNS efficacy in this sample.

### No Effect of Stimulation Device on Outcome Measures

Most participants completed the study using the Parasym taVNS device (n=20), while a small subset of participants completed the study using a TENS7000 device (n=4). To ensure that the stimulation device used did not impact the findings, we repeated the analyses in the participants that received stimulation from the Parasym device and replicated the pattern of results observed in the full sample. In this subsample, there was no benefit of active taVNS on reading accuracy (t (18)=0.13, p=0.90) or on reading rate (t (18)=0.17, p=0.87). With respect to reading comprehension performance, there was a trending benefit of active taVNS across all comprehension questions (t (18)=1.91, p=0.07), which was driven by a significant benefit on memory-based comprehension questions (t (18)=2.33, p=0.03). There was no group difference on inference-based comprehension questions (t (18)=0.19, p=0.86. This consistent pattern suggests that stimulation device differences did not impact the efficacy of posterior tragus stimulation of the auricular vagus nerve on our dependent measures.

### Relationships Between Verbal Working Memory and Comprehension

Based on prior evidence that comprehension abilities require working memory skills (Cain, Oakhill, & Bryant, 2004), we next utilized Pearson's r to evaluate the relationships between verbal working memory and performance on memory-based comprehension questions. In the sham taVNS group, performance on the Verbal Learning Core (WRAIVIL-2; Sheslow & Adams, 2009) was not correlated to performance memory-based questions (r=0.05, p=0.88), but it was significantly and positively correlated within the active taVNS group, even after correction (r=0.65, p=0.023). In the sham taVNS group, performance on the Verbal Learning Recall (WRAML-2; Sheslow & Adams, 2009) was not correlated to performance on memory-based questions (r=−0.39, p=0.24), but there was a significant, positive relationship in the active taVNS group, even after correction (r=0.65, p=0.021).

### Discussion

The aim of the current study was to evaluate the effect of taVNS on a cognitive skill such as reading comprehension in a sample of typical young adult readers. Our results demonstrate, for the first time, that taVNS paired with reading improves performance on reading comprehension questions and that this benefit appears to be selective to memory-based recall. We observed no effect of taVNS on inference-based reading comprehension or the mechanics of oral reading (reading accuracy and reading rate). These findings support prior work demonstrating that taVNS is capable of improving reading skills (Thakkar et al., 2020). However, our findings also suggest that the efficacy of taVNS may be limited to tasks relying on sensory plasticity and memory.

### Vagus Nerve Stimulation Improves Memory

There is a well-documented literature of both cVNS and taVNS yielding significant results in both the animal model and in human participants (Engineer et al., 2011; De Ridder et al., 2013; Jacobs et al., 2015; Redgrave et al., 2018). When taVNS was investigated in the sensory and motor domains, significant benefits were found, suggesting that it can provide the same benefits as cVNS. For example, taVNS paired with sound therapy for ten sessions led to decreased tinnitus symptoms in a small sample of patients, unique for those specific sounds exposed during training (Lehtimaki et al., 2013). Further, taVNS paired with physical rehabilitation over 18 sessions also showed significant improvements in learning of trained movements in post-stroke participants. These findings provide additional evidence that VNS can boost memory, taking advantage of precisely timed neurotransmitter release. However, motor studies (Redgrave et al., 2018) do not test or acknowledge performance on other motor movements that were not taught.

In cognitive studies, older adults benefitted from taVNS during encoding and consolidation when learning to associate faces and names. In a single training session, active taVNS led to better associative memory performance than did sham taVNS (Jacobs et al., 2015). Similarly, key findings from the current study were unique to memory-based comprehension questions, with no taVNS benefit for inference-based comprehension questions. It is important to note that inference skills are essential in reading comprehension, since students must infer meaning from text to truly learn material. It is possible that taVNS can aid in inferential learning, but stimulation protocols may need to be altered such that stimulation is delivered during oral reading and a brief consolidation period. That approach of extending stimulation beyond just during oral reading may allow the brain to process text in a more efficient manner, and it more closely models the stimulation protocol in other studies (Clark et al., 1999; Jacobs et al., 2015).

### Comparison to Behavioral-Based Interventions

In the current study, we observed robust effects of taVNS on memory-based comprehension questions in a single session of stimulation. Previous interventions, such as Let's Know! (LARRC, 2015; LARRC & Chiu, 2018; LARRC, Pratt, & Logan, 2014) and randomized controlled trials (Clarke et al., 2009; LARRC; 2015) require multiple sessions per week for several weeks to produce significant benefits. Similar to previous interventions, reading comprehension was measured through standardized English assessments. However, there were crucial differences between previous interventions and the taVNS intervention used in the current study. First, previous interventions focused weeks- or months-long intervention on lower-level skills needed to improve reading comprehension, such as vocabulary learning (Clarke et al., 2009). In the current study, we attempted to bypass this long training time by pairing taVNS with a single session of real-time reading. While we observed significant benefits of taVNS in a single session, it is unknown whether these effects are short-lived or long-lived, as we did not evaluate the effect of taVNS on long-term reading comprehension skills in the days and weeks after the stimulation session. It is likely that long-term benefits could be observed, since evidence has been seen in both the rodent model up to three weeks after therapy (Engineer et al., 2011) and in the human model between three- and six-months post-therapy (De Ridder et al., 2013) for sensory plasticity. However, similar research has yet to be conducted using taVNS. The present disclosure can include and be advantageous in understanding comprehension and both short- and long-term memory, since students often must comprehend and store information for assessments (i.e., exams) that take place days or weeks after the material is taught in the classroom.

Second, while other classroom programs or interventions require multiple hours of training each week for many weeks (Clarke et al., 2009; Martins & Carnio, 2019), we were able to enhance comprehension on memory-related questions during a single session. Together, these differences suggest that stimulating the brain during reading can improve reading skills faster than using other strategies over many sessions. This difference is important because interventions that demand longer period of time can also be taxing for students, and it is possible that some students may be unable to complete a full intervention schedule. The addition of an approach like taVNS, which may shorten the time needed for intervention, could increase the likelihood that children are able to complete and benefit from the full program.

Third, previous interventions (e.g., Martins & Carnio, 2020) were applied in struggling readers and were validated after 16-weeks of targeted text reading and comprehension, finding that children were benefitting from intervention and experienced increased motivation. The current study was conducted on typically developing young adults, and it is currently unknown whether taVNS is effective in those with reading disorders. Individuals with dyslexia exhibit deficits in a variety of lower-level reading skills, such as decoding, phonological awareness, and automaticity (Pennington & Bishop, 2009; Wolf & Obregon, 1992). These deficits often lead to individuals focusing their cognitive resources on the mechanics of reading rather than on absorbing the content of a passage (cite if possible). In the current study, all the participants were typically developing readers and thus exhibited average or above average low-level reading skills. It is therefore possible that a taVNS intervention for dyslexia would require a focus on the mechanics of reading rather than on comprehension. Our prior work demonstrated a benefit of taVNS on letter-sound learning and subsequent improvement in automaticity and decoding (Thakkar et al., 2020). It is unknown whether this approach is also effective in dyslexia and our ongoing work is designed to investigate potential effects in individuals with dyslexia.

### Working Memory Skills Influence Reading Comprehension

With regard to baseline memory skills, a significant, positive correlation between verbal learning and recall and performance on memory-based comprehension questions emerged in the active taVNS group, but not the sham taVNS group. These significant correlations remained Executive functions, such as updating, shifting, and memory, each have been shown to contribute to reading comprehension (see Butterfuss & Kendeou, 2018, for a review). Working memory is essential for retaining and integrating information from text, which is a marker of understanding. Data from both children and adults have suggested that oral language and decoding skills mediate the relationship between working memory and reading comprehension (Spencer et al., 2020). Additional findings have corroborated these relationships between memory skills and reading comprehension with other verbal memory tasks (e.g., sentence span; Daneman & Carpenter, 1980) and in individuals with inadequate reading skills (see Peng et al., 2018 for a meta-analysis).

Prior studies have further elucidated the role of memory skills in reading comprehension. One meta-analysis reported that executive functions (e.g., reading span, counting span, digit span) were significantly correlated with reading comprehension, even after accounting for various variables, such as age or type of assessment used (Follmer, 2018). Specifically, individuals with higher working memory consistently had higher reading comprehension performance. Given the results from the current study, it is possible that taVNS takes advantage of high verbal memory to compound effects and increase reading comprehension. Additionally, since previous data have suggested that memory is still essential for comprehension in struggling readers, it is reasonable to speculate that our significant correlation can be replicated. In another embodiment, the present disclosure can include an effect of taVNS and verbal memory interactions in individuals with dyslexia.

No Effect of Current Intensity on taVNS Efficacy

Previous work investigating the impact of current intensity on cVNS efficacy in the rat model reported that moderate stimulation intensities were more effective than higher stimulation intensities (Borland et al., 2016). A similar effect was reported in humans such that moderate intensities were more effective than higher current intensities for improvement on a recognition memory task (Clark et al., 1999). Contrary to this evidence from the cVNS literature, we observed no relationship between taVNS current intensity and performance on comprehension. It is possible that taVNS exhibits a different dose-response curve with respect to current intensity compared to invasive vagus nerve stimulation. In support of this hypothesis, prior research has revealed benefits of taVNS at a variety of intensities. One study reported that taVNS delivered at intensities below sensory threshold can selectively enhance speech sound categorizations (Llanos et al., 2020). Other studies conducted above sensory threshold reported a benefit of taVNS in cognitive tasks, such as face-name association and novel letter-sound learning (Thakkar et al., 2020). However, it is important to note that these prior studies did not manipulate current intensity or systematically investigate potential relationships between current intensity and outcome measures. The present disclosure can include influence of current intensity on outcome measures. Such findings could inform on ideal parameters to consistently enhance performance and drive plasticity.

### Limitations

There are three limitations in the current study. First, most of the passages used for testing included a high number of low-frequency words. Given the added pressure of reading out loud, participants may have spent more cognitive effort focusing on decoding of these words rather than processing meaning. Findings from such a study would suggest that readers, when not pressured to focus on decoding low-frequency words, can still maintain enhanced comprehension when paired with taVNS.

Second, all participants were from high SES backgrounds, stunting our ability to generalize these findings. Previous research suggests that home environment and SES play a role in future reading ability (Bowey, 1995; Bradley & Corwyn, 2002; Cheng & Wu, 2017; Kieffer, 2010). Specifically, children from a low SES background have more phonological deficits than those from a higher SES, and phonological skills are necessary for successful reading (Bowey, 1995). The present disclosure can include a sample that comes from a wider range of SES to determine whether the biological impact of a lower SES upbringing impact the efficacy of taVNS. Additionally, the sample should include a group of participants with dyslexia, as struggling readers have been shown to have impaired reading comprehension performance compared to their typically reading peers (Simmons & Singleton, 2000).

Third, our sample size is small due to the forced discontinuation of in-person research during the COVID-19 pandemic. While significant effects were found, the current sample did not meet the initial a priori power analysis (Faul et al., 2007). A post-hoc analysis (d=1.23, a=0.05, nₛₕₐₘ=12, n_{active}=12) yielded a power of 0.82.

### REFERENCES FOR EXAMPLE #1

Cohen L, Lehericy S, Chochon F, Lemer C, Rivaud S, Dehaene S. Languagespecific tuning of visual cortex? Functional properties of the visual word form area. Brain 2002; 125:1054e69. https://doi.org/10.1093/brain/awf094.
Dehaene S, Cohen L. Cultural recycling of cortical maps. Neuron 2007; 56: 384e98. https://doi.org/10.1016/j.neuron.2007.10.004.
Centanni T M, King L W, Eddy M D, Whitfield-Gabrieli S, Gabrieli J D E. Development of sensitivity and specificity for print in the visual word form area. Brain Lang 2017; 170:62e70. https://doi.org/10.1016/j.band1.2017.03.009.
Abadzi H. Does age diminish the ability to learn fluent reading? Educ Psychol Rev 1996; 8:373e95. https://doi.org/10.1007/BF01463940.
Abadzi H. Can adults become fluent readers in newly learned scripts? Educ Res Int 2012:710785. https://doi.org/10.1155/2012/710785.
Christodoulou J A, Del Tufo S N, Lymberis J, Saxler P K, Ghosh S S, Triantafyllou C, et al. Brain bases of reading fluency in typical reading and impaired fluency in dyslexia. PloS One 2014; 9(7):e100552. https://doi.org/10.1371/journal.pone.0100552.
Wolf M, Katzir-Cohen T. Reading fluency and its intervention. Sci Stud Read 2001; 5(3):211e39. https://doi.org/10.1207/51532799XSSR0503_2.
Picciotto M R, Higley M J, Mineur Y S. Acetylcholine as a neuromodulator: cholinergic signaling shapes nervous system function and behavior. Neuron 2012; 76(1):116e29. https://doi.org/10.1016/j.neuron.2012.08.036.
Tully K, Volshakov V Y. Emotional enhancement of memory: how norepinephrine enables synaptic plasticity. Mol Brain 2010; 3:15. https://doi.org/10.1186/1756-6606-3-15.
Maity S, Rah S, Sonenberg N, Gkogkas C G, Nguyen P V. Norepinephrine triggers metaplasticity of LTP by increasing translation of specific mRNAs. Learn Mem 2015; 22:499e508. https://doi.org/10.1101/lm.039222.115.
Glennon E, Carcea I, Martins A R O, Multani J, Shehu I, Svirsky M A, Froemke. Locus coeruleus activation accelerates perceptual learning. Brain Res 2019; 1709:39e49. https://doi.org/10.1016/j.brainres.2018.05.048.
Hulsey D R, Shedd C M, Sarker S F, Kilgard M P, Hays S A. Norepinephrine and serotonin are required for vagus nerve stimulation directed cortical plasticity. Exp Neurol 2019; 320:112975. https://doi.org/10.1016/j.expneurol.2019.112975.
Hulsey D R, Hays S A, Khodaparast N, Ruiz A, Das P, Rennaker R L, et al. Reorganization of motor cortex by vagus nerve stimulation requires cholinergic innervation. Brain Stimul 2016; 9(2):174e81. https://doi.org/10.1016/j.brs.2015.12.007.
Clewett D, Schoeke A, Mather M. Locus coeruleus neuromodulation of memories encoded during negative or unexpected action outcomes. Neurobiol Learn Mem 2014; 111:65e70. https://doi.org/10.1016/j.nlm.2014.03.006.
DeGiorgio C M, Schachter S C, Handforth A, Salinsky M, Thompson J, Uthman B, et al. Prospective long-term study of vagus nerve stimulation for the treatment of refractory seizures. Epilepsia 2000; 41(9):1195e200. https://doi.org/10.1111/j.1528-1157.2000.tb00325.x.
Morris G L, Mueller W. Vagus Nerve Stimulation Study Group. Long-term treatment with vagus nerve stimulation in patients with refractory epilepsy. Neurology 1999; 53(8):1731e5. https://doi.org/10.1212/wnl.53.8.1731.
Sackeim H A, Rush A J, George M S, Marangell L B, Husain M M, Nahas Z, et al. Vagus nerve stimulation (VNS) for treatment-resistant depression: efficacy, side effects, and predictors of outcome. Neuropsychopharmacology 2001; 25(5):713e28. https://doi.org/10.1016/50893-133X(01)00271-8.
Dawson J, Pierce D, Dixit A, Kimberley T J, Robertson M, Tarver B, et al. Safety, feasibility, and efficacy of vagus nerve stimulation paired with upper-limb rehabilitation after ischemic stroke. Stroke 2016; 47:143e50. https://doi.org/10.1161/strokeaha.115.010477.
De Ridder D, Vanneste S, Engineer N D, Kilgard M P. Safety and efficacy of vagus nerve stimulation paired with tones for the treatment of tinnitus: a case series. Neuromodulation 2014; 17:170e9. https://doi.org/10.1111/ner.12127.
Borland M S, Vrana W A, Moreno N A, Fogarty E A, Buell E P, Sharma P, et al. Cortical map plasticity as a function of vagus nerve stimulation intensity. Brain Stimul 2016; 9(1): 117e23. https://doi.org/10.1016/j.brs.2015.08.018.
Engineer N D, Riley J R, Seale J D, Vrana W A, Shetake J A, Sudanagunta S P, et al. Reversing pathological neural activity using targeted plasticity. Nature 2011; 470:101e6. https://doi.org/10.1038/nature09656.
Shetake J A, Engineer N D, Vrana W A, Wolf J T, Kilgard M P. Pairing tone trains with vagus nerve stimulation induces temporal plasticity in auditory cortex. Exp Neurol 2012; 233:342e9. https://doi.org/10.1016/j.expneurol.2011.10.026.
Khodaparast N, Hays S A, Sloan A M, Hulsey D R, Ruiz A, Pantoja R L, et al. Vagus nerve stimulation during rehabilitative training improves forelimb strength following ischemic stroke. Neurobiol Dis 2013; 60:80e8. https://doi.org/10.1016/j.nbd.2013.08.002.
Khodaparast N, Hays S A, Sloan A M, Fayyaz T, Hulsey D R, Rennaker R L, et al. Vagus nerve stimulation delivered during motor rehabilitation improves recovery in a rat model of stroke. Neurorehabilitation Neural Repair 2014; 28(7): 698e706. https://doi.org/10.1177/1545968314521006.
Porter B A, Khodaparast N, Fayyaz T, Cheung R J, Ahmed S S, Vrana W A, et al. Repeatedly pairing vagus nerve stimulation with a movement reorganizes primary motor cortex. Cerebr Cortex 2012; 22(10):2365e74. https://doi.org/10.1093/cercor/bhr316.
Clark K B, Naritoku D K, Smith D C, Browning R A, Jensen R A. Enhanced recognition memory following vagus nerve stimulation in human subjects. Nat Neurosci 1999; 2(1):94e8. https://doi.org/10.1038/4600.
Sun L, Perakyla J, Holm K, Haapasalo J, Lehtimaki K, Ogawa K H, et al. Vagus nerve stimulation improves working memory performance. J Clin Exp Neuropsychol 2017; 39(10):954e64. https://doi.org/10.1080/13803395.2017.1285869.
Sackeim H A, Keilp J G, Rush A J, George M S, Marangell L B, Dormer J S, et al. The effects of vagus nerve stimulation on cognitive performance in patients with treatment-resistant depression. Neuropsychiatry, Neuropsychology, and Behavioral Neurology 2001; 14(1):53e62.
Frangos E, Ellrich J, Komisaruk B R. Non-invasive access to the vagus nerve central projections via electrical stimulation of the external ear: fMRI evidence in humans. Brain Stimul 2015; 8(3):624e36. https://doi.org/10.1016/j.brs.2014.11.018.
Redgrave J N, Moore L, Oyekunle T, Ebrahim M, Falidas K, Snowdon N, et al. Transcutaneous auricular vagus nerve stimulation with concurrent upper limb repetitive task practice for post stroke motor recovery: a pilot study. J Stroke Cerebrovasc 2018; 27(7):1998e2005. https://doi.org/10.1016/j.jstrokecerebrovasdis.2018.02.056.
Yakunina N, Kim S S, Nam E-C. Optimization of transcutaneous vagus nerve stimulation using functional M M. Neuromodulation 2016; 20(3):290e300. https://doi.org/10.1111/ner. 12541.
Badran B W, Dowdle L T, Mithoefer O J, LaBate N T, Coatsworth J, Brown J C, et al. Neurophysiologic effects of transcutaneous auricular vagus nerve stimulation (taVNS) via electrical stimulation of the tragus: a concurrent taVNS/fMRI study and review. Brain Stimul 2018; 11(3):492e500. https://doi.org/10.1016/j.brs0.2017.12.009.
Jacobs H I L, Riphagen J M, Razat C M, Wiese S, Sack A T. Transcutaneous vagus nerve stimulation boosts associative memory in older individuals. Neurobiol Aging 2015; 36:1860e7. https://doi.org/10.1016/j.neurobiolaging.2015.02.023.
Kaufman A S, Kaufman N L. KBIT-2 Kaufman brief intelligence test. second ed. Bloomington, M N: Pearson; 2004.
Torgesen J, Wagner R, Rashotte C. Test of word reading efficiency. second ed. Austin, Tex.: ProEd, Inc; 2012.
Woodcock R W. Woodcock reading mastery tests. third ed. Bloomington, Minn.: American Guidance Service Circle Pines; 2011.
Wagner R K, Torgesen J K, Rashotte C A. CTOPP-2: comprehensive test of phonological processing. second ed. Austin, Tex.: ProEd, Inc; 2013.
Sheslow D, Adams W. Wide range assessment of memory and learning. second ed. Austin, Tex.: ProEd, Inc; 2009.
Brown G L, Eccles J C. The action of a single vagal volley on the rhythm of the heartbeat. J Physiol 1934; 82(2):211e41.
Muppidi S, Gupta P K, Vernino S. Reversible right vagal neuropathy. Neurology 2011; 77:1577e9.
Kaniusas E, Kampusch S, Tittgemeyer M, Panetsos F, Gines R F, Papa M, et al. Current directions in the auricular vagus nerve stimulation I-A physiological perspective. Front Neurosci 2019; 13:854. https://doi.org/10.3389/fnins.2019.00854.
Kaniusas E, Kampusch S, Tittgemeyer M, Panetsos F, Gines R F, Papa M, et al. Current directions in the auricular vagus nerve stimulation II—an engineering perspective. Front Neurosci 2019; 13:772. https://doi.org/10.3389/fnins.2019.00772.
Peirce J W, Gray J R, Simpson S, MacAskill M R, Hochenberger R, Sogo H, et al. PsychoPy2: experiments in behavior made easy. Behav Res Methods 2019; 51(1):195e203. https://doi.org/10.3758/s13428-018-01193-y.
Abadzi H, Prouty R. Discerning shapes, reading words like faces: the current science of literacy and its implications for low-income countries. Int Forum 2012; 15(1):5e28.
Norton E S, Wolf M. Rapid automatized naming (RAN) and reading fluency: implications for understanding and treatment of reading disabilities. Annu Rev Psychol 2012; 63:427e52. https://doi.org/10.1146/annurev-psych120710-100431.
Lervag A, Hulme C. Rapid automatized naming (RAN) taps a mechanism that places constraints on the development of early reading fluency. Psychol Sci 2009; 20(8):1040e8. https://doi.org/10.1111/j 0.1467-9280.2009.02405.x.
Centanni T M, Pantazis D, Truong D T, Gruen J R, Gabrieli J D E, Hogan T P. Increased variability of stimulus-driven cortical responses is associated with genetic variability in children with and without dyslexia. Dev Cogn Neuros 2018; 34:7e17. https://doi.org/10.1016/j.dcn.2018.05.008.
Martin L, Durisko C, Moore M W, Coutanche M N, Chen D, Fiez J A. The VWFA is the home of orthographic learning when houses are used as letters. eNeuro 2019; 6(1):e0425. https://doi.org/10.1523/ENEURO.0425-17.2019.17.
Moore M W, Durisko C, Perfetti C A, Fiez J A. Learning to read an alphabet of human faces produces left-lateralized training effects in the fusiform gyms. J Cognit Neurosci 2014; 26(4):896e913. https://doi.org/10.1162/jocn_a_00506.
Nielson K. Self-study with language learning software in the work place: what happens? Lang Learn Technol 2011; 15(3):110e29.
Straube A, Ellrich J, Eren O, Blum B, Ruscheweyh R. Treatment of chronic migraine with transcutaneous stimulation of the auricular branch of the vagal nerve (auricular t-VNS): a randomized, monocentric clinical trial. J Headache Pain 2015; 16:63. https://doi.org/10.1186/s10194-015-0543-3.
Clarencon D, Pellissier S, Sinniger V, Kibleur A, Hoffman D, Vercueil L, et al. Long term effects of low frequency (10 Hz) vagus nerve stimulation on EEG and heart rate variability in Crohn's disease: a case report. Brain Stimul 2014; 7: 914e5. https://doi.org/10.1016/j.brs.2014.08.001.
Llanos F, McHaney J R, Schuerman W L, Yi H G, Leonard M K, Chandrasekaran B. Non-invasive peripheral nerve stimulation selectively enhances speech category learning in adults. NPJ Sci 2020; 5:12. https://doi.org/10.1038/s41539-020-0070-0.

### REFERENCES FOR EXAMPLE #2

Archibald, L. M. D. (2017). Working memory and language learning: A review. Child Language Teaching and Therapy, 33(1), 5-17. https://doi.org/10.1177/0265659016654206
Atkins, P. W. B., & Baddeley, A. D. (1998). Working memory and distributed vocabulary learning. Applied Psycholinguistics, 19, 537-552. https://doi.org/10.1017/S0142716400010353
Badran, B. W., Dowdle, L. T., Mithoefer, O. J., LaBate, N. T., Coatsworth, J., Brown, J. C., DeVries, W. H., Austelle, C. W., McTeague, L. M., & George, M. S. (2018). Neurophysiologic effects of transcutaneous auricular vagus nerve stimulation (taVNS) via electrical stimulation of the tragus: A concurrent taVNS/fMRI study and review. Brain Stimulation, 11, 492-500. https://doi.org/10.1016/j.brs.2017.12.009
Bahrick, H. P., & Phelps, E. (1987). Retention of Spanish vocabulary over 8 years. Journal of Experimental Psychology: Learning, Memory, and Cognition, 13(2), 344-349. https://doi.org/10.1037/0278-7393.13.2.344
Bartolotti, J., Bradley, K., Hernandez, A. E., & Marian, V. (2017). Neural signatures of second language learning and control. Neuropsychologia, 98, 130-138. https://doi.org/10.1016/j.neuropsychologia.2016.04.007
Bialystok, E., Craik, F. I. M., & Freedman, M. (2007). Bilingualism as a protection against the onset of symptoms of dementia. Neuropsychologia, 45, 459-464. doi:10.1016/j.neuropsychologia.2006.10.009
Birdsong, D. (2018). Plasticity, variability and age in second language acquisition and bilingualism. Frontiers in Psychology, 9, 81. https://doi.org/10.3389/fpsyg.2018.00081
Bialystok, E., Craik, F. I. M., & Luk, G. (2012). Bilingualism: Consequences for mind and brain. Trends in Cognitive Sciences, 16(4), 240-250. doi:10.1016/j.tics.2012.03.001
Birdsong, D., & Molis, M. (2001). On the evidence for maturational constraints in second-language acquisition. Journal of Memory and Language, 44(2), 235-249. doi: 10.1006/jmla.200.2750
Bishop, D. V. M., Barry, J. G., & Hardiman, M. J. (2012). Delayed retention of new word-forms is better in children than adults regardless of language ability: A two factorial two-way study. PLoS ONE, 7(5), e37326. doi:10.1371/journal.pone.0037326
Borland, M. S., Vrana, W. A., Moreno, N. A., Fogarty, E. A., Buell, E. P., Sharma, P., Engineer, C. T., & Kilgard, M. P. (2016). Cortical map plasticity as a function of vagus nerve stimulation intensity. Brain Stimulation, 9, 117-123. http://dx.doi.org/10.1016/j.brs.2015.08.018
Brown, H., Weighall, A., Henderson, L. M., & Gaskell, M. G. (2012). Enhanced recognition and recall of new words in 7- and 12-year-olds following a period of offline consolidation. Journal of Experimental Child Psychology, 112, 56-72. https://doi.org/10.1016/j.jecp.2011.11.010
Butt, M. F., Albusoda, A., Farmer, A. D., & Aziz, Q. (2020). The anatomic basis for transcutaneous auricular vagus nerve stimulation. Journal of Anatomy, 236, 588-611. doi: 10.1111/joa.13122
Clark, K. B., Naritoku, D. K., Smith, D. C., Browning, R. A., & Jensen, R. A. (1999). Enhanced recognition memory following vagus nerve stimulation in human subjects. Nature Neuroscience, 2(1), 94-98. doi: 10.1038/4600
Colzato, L., & Beste, C. (2020). A literature review on the neurophysiological underpinnings and cognitive effects of transcutaneous vagus nerve stimulation: Challenges and future directions. Journal of Neurophysiology, 123, 1739-1755. https://doi.org/10.1152/jn.00057.2020
David, A., & Wei, L. (2008). Individual differences in the lexical development of French-English bilingual children. International Journal of Bilingual Education and Bilingualism, 11(5),
Dawson, J., Pierece, D., Dixit, A., Kimberley, T. J., Robertson, M., Tarver, B., . . . , & Engineer, N. (2016). Safety, feasibility, and efficacy of vagus nerve stimulation paired with upper-limb rehabilitation after ischemic stroke. Stroke, 47, 143-150. doi: 10.1161/strokeaha.115.010477
DeGiorgio, C. M., Schachter, S. C., Handforth, A., Salinsky, M., Thompson, J., Uthman, B., . . . , & Heck, C. (2000). Prospective long-term study of vagus nerve stimulation for the treatment of refractory seizures. Epilepsia, 41(9), 1195-1200. https://doi.org/10.1111/j 0.1528-1157.2000.tb00325.x
De Ridder, D., Kilgard, M., Engineer, N., & Vanneste, S. (2015). Placebo-controlled vagus nerve stimulation paired with tones in a patient with refractory tinnitus: A case report. Otology & Neurotology, 36, 575-580. doi: 10.1097/MAO.0000000000000704
Engineer, N. D., Riley, J. R., Seale, J. D., Vrana, W. A., Shetake, J. A., Sudanagunta, S. P., Borland, M. S., & Kilgard, M. P. (2011). Reversing pathological neural activity using targeted plasticity. Nature, 470, 101-106. doi:10.1038/nature09656
Faul, F., Erdfelder, E., Lang, A., & Buchner, A. (2007). G*Power 3: A flexible statistical power analysis program for the social, behavioral, and biomedical sciences. Behavior Research Methods, 39(2), 175-191.
Fernald, A., Marchman, V. A., & Weisleder, A. (2013). SES differences in language processing skill and vocabulary are evident at 18 months. Developmental Science, 16(2), 234-248. doi: 10.1111/desc.12019
Fiori, V., Kunz, L., Kuhnke, P., Marangolo, P., & Hartwigsen, G. (2018). Transcranial direct current stimulation (tDCS) facilitates verb learning by altering effective connectivity in the healthy brain. NeuroImage, 181, 550-559. https://doi.org/10.1016/j.neuroimage.2018.07.040
Fliessbach, K., Weis, S., Klaver, P., Elger, C. E., & Weber, B. (2006). The effect of word concreteness on recognition memory. NeuroImage, 32, 1413-1421. doi:10.1016/j.neuroimage.2006.06.007
Floel, A., Rosser, N., Michka, O., Knecht, S., & Breitenstein, C. (2008). Noninvasive brain stimulation improves language learning. Journal of Cognitive Neuroscience, 20(8), 1415-1422. doi: 10.1162/jocn.2008.20098
Frangos, E., Ellrich, J., & Komisaruk, B. R. (2015). Non-invasive access to the vagus nerve central projections via electrical stimulation of the external ear: fMRI evidence in humans. Brain Stimulation, 8(3), 624-636. doi: 10.1016/j.brs.2014.11.018
Gathercole, S. E., Hitch, G. J., Service, E., & Martin, A. J. (1997). Phonological short-term memory and new word learning in children. Developmental Psychology, 33(6), 966-979. doi: 10.1037//0012-1649.33.6.966
Giraudier, M., Ventura-Bort, C., & Weymar, M. (2020). Transcutaneous vagus nerve stimulation (tVNS) improves high-confidence recognition memory but not emotional word processing. Frontiers in Psychology, 11, 1276. https://doi.org/10.3389/fpsyg.2020.01276
Groves, D. A. & Brown, V. J. (2005). Vagal nerve stimulation: A review of its applications and potential mechanisms that mediate its clinical effects. Neuroscience and Biobehavioral Reviews, 29, 493-500. doi:10.1016/j.neubiorev.2005.01.004
Gupta, P., & MacWhinney, B. (1997). Vocabulary acquisition and verbal short-term memory: Computational and neural bases. Brain and Language, 59, 267-333. doi: 10.1006/brin.1997.1819
Hamilton, M., & Rajaram, S. (2001). The concreteness effect in implicit and explicit memory tests. Journal of Memory and Language, 44(1), 96-117. https://doi.org/10.1006/jmla.2000.2749
Hansson, K., Forsberg, J., Lofqvist, A., Maki-Torkko, E., & Sahlen, B. (2004). Working memory and novel word learning in children with hearing impairment and children with specific language impairment. International Journal of Language & Communication Disorders, 39(3), 401-422. doi: 10.1080/13682820410001669887
Hartshorne, J. K., Tenenbaum, J. B., & Pinker, S. (2018). A critical period for second language acquisition: Evidence from 2/3 million English speakers. Cognition, 177, 263-277. https://doi.org/10.1016/j.cognition.2018.04.007
Heinze, S., Sartory, G., Müller, B. W., de Greiff, A., Forsting, M., & Jüptner, M. (2006). Neural encoding correlates of high and low verbal memory performance. Journal of Psychophysiology, 20(2), 68-78. https://doi.org/10.1027/0269-8803.20.2.68
Hulsey, D. R., Hays, S. A., Khodaparast, N., Ruiz, A., Das, P., Rennaker, R. L., & Kilgard, M. P. (2016). Reorganization of motor cortex by vagus nerve stimulation requires cholinergic innervation. Brain Stimulation, 9, 174-181. http://dx.doi.org/10.1016/j.brs.2015.12.007
Hurford, J. R. (1991). The evolution of the critical period for language acquisition. Cognition, 40, 159-201. https://doi.org/10.1016/0010-0277(91)90024-X
Jacobs, H. I. L., Riphagen, J. M., Razat, C. M., Wiese, S., & Sack, A. T. (2015). Transcutaneous vagus nerve stimulation boosts associative memory in older individuals. Neurobiology of Aging, 36, 1860-1867. http://dx.doi.org/10.1016/j.neurobiolaging.2015.02.023
Jarrold, C., Thorn, A. S. C., & Stephens, E. (2009). The relationships among verbal short-term memory, phonological awareness, and new word learning: Evidence from typical development and Down syndrome. Journal of Experimental Child Psychology, 102, 196-218. https://doi.org/10.1016/j.jecp.2008.07.001
Jenkins, J. R., Stein, M. R., & Wysocki, K. (1984). Learning vocabulary through reading. American Educational Research Journal, 21, 767-787.
Johnson, J. S., & Newport, E. L. (1989). Critical period effects in second language learning: The influence of maturational state on the acquisition of English as a second language. Cognitive Psychology, 21, 60-99. https://doi.org/10.1016/0010-0285(89)90003-0
Kaufman, A. S. & Kaufman, N. L. (2004). K-BIT: Kaufman brief intelligence test. (2nd ed.). Bloomington, Minn.: Pearson.
Kaushanskaya, M., & Marian, V. (2009). The bilingual advantage in novel word learning. Psychonomic Bulletin & Review, 16(4), 705-710. https://doi.org/10.3758/PBR. 16.4.705
Khodaparast, N., Hays, S. A., Sloan, A. M., Hulsey, D. R., Ruiz, A., Pantoja, R. L., & Kilgard, M. P. (2013). Vagus nerve stimulation during rehabilitative training improves forelimb strength following ischemic stroke. Neurobiology of Disease, 60, 80-88. doi: 10.1016/j.nbd.2013.08.002
Kim, Y-S. G. (2017). Multicomponent view of vocabulary acquisition: An investigation with primary grade children. Journal of Experimental Child Psychology, 162, 120-133. http://dx.doi.org/10.1016/j.jecp.2017.05.004
Kong, J., Fang, J., Park, J., Li, S., & Rong, P. (2018). Treating depression with transcutaneous auricular vagus nerve stimulation: State of the art and future perspectives. Frontiers in Psychiatry, 9, 20. doi: 10.3389/fpsyt.2018.00020
Krishnan, S., Watkins, K. E., & Bishop, D. V. M. (2017). The effect of recall, reproduction, and restudy on word learning: A pre-registered study. BMC Psychology, 5, 28. 10.1186/s40359-017-0198-8
Lenneberg, E. (1967). Biological foundations of language. New York, N.Y.: Wiley. Llanos, F., McHaney, J. R., Schuerman, W. L., Yi, H. G., Leonard, M. K., & Chandrasekaran, B. (2020). Non-invasive peripheral nerve stimulation selectively enhances speech category learning in adults. NPJ Science of Learning, 5, 12. https://doi.org/10.1038/s41539 0070-0
Loewen, S., Crowther, D., Isbell, D. R., Kim, K. M., Maloney, J., Miller, Z. F., & Rawal, H. (2019). Mobile-assisted language learning: A Duolingo case study. ReCALL, 31(3), 293-311. doi:10.1017/S0958344019000065
Long, M. H. (1990). Maturational constraints on language development. Studies in Second Language Acquisition, 12, 251-285. https://doi.org/10.1017/S0272263100009165
Lu, M. (2008). Effectiveness of vocabulary learning via mobile phone. Journal of Computer Assisted Learning, 24, 515-525. https://doi.org/10.1111/j.1365-2729.2008.00289.x
Majerus, S. (2013). Language repetition and short-term memory: An integrative framework. Frontiers in Human Neuroscience, 7, 357. doi: 10.3389/fnhum.2013.00357
McGregor, K. K., Gordon, K., Eden, N., Arbisi-Kelm, T., & Oleson, J. (2017). Encoding deficits impede word learning and memory in adults with developmental language disorders. Journal of Speech, Language, and Hearing Research, 60, 2891-2905. https://doi.org/10.1044/2017_JSLHR-L-17-0031
McManus, W., Gould, W., & Welch, F. (1983). Earnings of Hispanic men: The role of English language proficiency. Journal of Labor Economics, 1(2), 101-130. doi: 10.1086/298006
Medeiros, L. F., Custodio de Souza, I. C., Vidor, L. P., de Souza, A., Deitos, A., Volz, M. S., Fregni, F., Caumo, W., & Tones, I. L. S. (2012). Neurobiological effects of transcranial direct current stimulation: A review. Frontiers in Psychiatry, 3, 110. doi: 10.3389/fpsyt.2012.00110
Mertens, A., Naert, L., Miatton, M., Poppa, T., Carrette, E., Gadeyne, S., Raedt, R., Boon, P., & Vonck, K. (2020). Transcutaneous vagus nerve stimulation does not affect verbal memory performance in healthy volunteers. Frontiers in Psychology, 11, 551. https://doi.org/10.3389/fpsyg.2020.00551
Morris, G. L., Mueller, W., & Vagus Nerve Stimulation Study Group. (1999). Long-term treatment with vagus nerve stimulation in patients with refractory epilepsy. Neurology, 53(8), 1731-1735. doi: 10.1212/wnl.53.8.1731
Morrison, R. A., Danaphongse, T. T., Abe, S. T., Stevens, M. E., Ezhil, V., Seyedahmadi, A., Adcock, K. S., Rennaker, R. L., Kilgard, M. P., & Hays, S. A. (2021). High intensity VNS disrupts VNS-mediated plasticity in motor cortex. Brain Research, 1756, 147332. https://doi.org/10.1016/j.brainres.2021.147332
Morrison, R. A., Danaphongse, T. T., Pruitt, D. T., Adcock, K. S., Mathew, J. K., Abe, S. T., Abdulla, D. M., Rennaker, R. L., Kilgard, M. P., & Hays, S. A. (2020). A limited range of vagus nerve stimulation intensities produce motor cortex reorganization when delivered during training. Behavioural Brain Research, 391, 112705. https://doi.org/10.1016/j.bbr.2020.112705
Newcombe, F. (1969). Missile wounds of the brain: A study of psychological deficits. Oxford University Press.
Nielson, K. B. (2011). Self-study with language learning software in the workplace: What happens? Language Learning & Technology, 15(3), 110-129.
Pasqualotto, A., Kobanbay, B., & Proulx, M. J. (2015). Neural stimulation has a long-term effect on foreign vocabulary acquisition. Neural Plasticity, 671705. https://doi.org/10.1155/2015/671705
Peirce, J., Gray, J. R., Simpson, S., MacAskill, M., Hochenberger, R., Sogo, H., Kastman, E., & Lindelov, J. K. (2019). PsychoPy2: Experiments in behavior made easy. Behavior Research Methods, 51(1), 195-203. https://doi.org/10.3758/s13428-018-01193-y
Perceval, G., Martin, A. K., Copland, D. A., Laine, M., & Meinzer, M. (2017). High-definition tDCS of the temporo-parietal cortex enhances access to newly learned words. Scientific Reports, 7, 17023. doi: 10.1038/s41598-017-17279-0
Perkins, S. C., Finegood, E. D., & Swain, J. E. (2013). Poverty and language development: Roles of parenting and stress. Innovations in Clinical Neuroscience, 10(4), 10-19.
Porter, B. A., Khodaparast, N., Fayyaz, T., Cheung, R. J., Ahmed, S. S., Vrana, W. A., Rennaker, R. L., & Kilgard, M. P. (2012). Repeated pairing vagus nerve stimulation with a movement reorganizes primary motor cortex. Cerebral Cortex, 22, 2365-2374. doi:10.1093/cercor/bhr316
Redgrave, J. N., Moore, L., Oyekunle, T., Ebrahim, M., Falidas, K., Snowdon, N., Ali, A., & Majid, A. (2018). Transcutaneous auricular vagus nerve stimulation with concurrent upper limb repetitive task practice for post stroke motor recovery: A pilot study. Journal of Stroke and Cerebrovascular Diseases, 27(7), 1998-2005. https://doi.org/10.1016/j.jstrokecerebrovasdis.2018.02.056
Sackeim, H. A., Rush, A. J., George, M. S., Marangell, L. B., Husain, M. M., Nahas, Z., Johnson, C. R., Seidman, S., Giller, C., Haines, S., Simpson, R. K., & Goodman, R. R. (2001). Vagus nerve stimulation (VNS) for treatment-resistant depression: Efficacy, side effects, and predictors of outcome. Neuropsychopharmacology, 25(5), 713-728. doi: 10.1016/S0893-133X(01)00271-8
Sackeim, H. A., Keilp, J. G., Rush, A. J., George, M. S., Marangell, L. B., Dormer, J. S., Burt, T., Lisanby, S. H., Husain, M., Cullum, C. M., Oliver, N., & Zboyan, H. (2001). The effects of vagus nerve stimulation on cognitive performance in patients with treatment-resistant depression. Neuropsychiatry, Neuropsychology, and Behavioral Neurology, 14(1), 53-62.
Salavati, M., & Salehi, H. (2016). Impact of using instructional video games as an on EFL learners' vocabulary retention. Universal Journal of Educational Research, 4(12), 2724-2728. doi: 10.13189/ujer.2016.041205
Sheslow, D. & Adams, W. (2009). Wide range assessment of memory and learning. (2nd ed.). Austin, Tex.: ProEd, Inc.
Shetake, J. A., Engineer, N. D., Vrana, W. A., Wolf, J. T., & Kilgard, M. P. (2012). Pairing tone trains with vagus nerve stimulation induces temporal plasticity in auditory cortex. Experimental Neurology, 233, 342-349. doi:10.1016/j.expneurol.2011.10.026
Straube, A., Ellrich, J., Eren, O., Blum, B., & Ruscheweyh, R. (2015). Treatment of chronic migraine with transcutaneous stimulation of the auricular branch of the vagal nerve (auricular t-VNS): A randomized, monocentric clinical trial. The Journal of Headache and Pain, 16, 63. doi: 10.1186/s10194-015-0543-3
Stefan, H., Kreiselmeyer, G., Kerling, F., Kurzbuch, K., Rauch, C., Heers, M., Kasper, B. S., Hammen, T., Rzonsa, M., Pauli, E., Ellrich, J., Graf, W., & Hopfengartner, R., (2012). Transcutaneous vagus nerve stimulation (t-VNS) in pharmacoresistant epilepsies: A proof of concept trial. Epilepsia, 53(7), e115-e118. doi: 10.1111/j.1528-1167.2012.03492.x
Sun, L., Perakyla, J., Holm, K., Haapasalo, J., Lehtimaki, K., Ogawa, K. H., Peltola, J., & Hartikainen, K. M., (2017). Vagus nerve stimulation improves working memory performance. Journal of Clinical and Experimental Neuropsychology, 39(10), 954-964. doi: 10.1080/13803395.2017.1285869
Thakkar, V. J., Engelhart, A. S., Khodaparast, N., Abadzi, H., & Centanni, T. M. (2020). Transcutaneous auricular vagus nerve stimulation enhances learning of novel letter-sound relationships in adults. Brain Stimulation, 13(6), 1813-1820. https://doi.org/10.1016/j.brs.2020.10.012
Torgesen, J., Wagner, R., & Rashotte, C. (2012). Test of word reading efficiency (2nd ed.). Austin, Tex.: ProEd, Inc.
Van den Noort, M., Vermeire, K., Bosch, P., Staudte, H., Krajenbrink, T., Jaswetz, L., Struys, E., Yeo, S., Barisch, P., Perriard, B., Lee, S-H., & Lim, S. (2019). A systemic review on the possible relationship between bilingualism, cognitive decline, and the onset of dementia. Behavioral Sciences, 9(7), 81. https://doi.org/10.3390/bs9070081
Vanhove, J. (2013). The critical period hypothesis in second language acquisition: A statistical critique and reanalysis. PLoS ONE, 8, e69172. https://doi.org/10.1371/journal.pone.0069172
Van Leusden, J. W. R., Sellaro, R., & Colzato, L. S. (2015). Transcutaneous vagal nerve stimulation (tVNS): A new neuromodulation tool in healthy humans? Frontiers in Psychology, 6, 102. https://doi.org/10.3389/fpsyg.2015.00102
Verhagen, J., & Leseman, P. (2016). How do verbal short-term memory and working memory relate to the acquisition of vocabulary and grammar? A comparison between first and second language learners. Journal of Experimental Child Psychology, 141, 65-82. http://dx.doi.org/10.1016/j.jecp.2015.06.015
Wagner, R. K., Torgesen, J. K., & Rashotte, C. A. (2013). CTOPP-2: Comprehensive test of phonological processing. (2nd ed.). Austin, Tex.: ProEd, Inc.
Wojcik, E. H. (2013). Remembering new words: Integrating early memory development into word learning. Frontiers in Psychology, 4, 151. https://doi org/10.3389/fpsyg.2013.00151
Woodcock, R. W. (2011). Woodcock reading mastery tests. (3rd ed.). Bloomington, Minn.: American Guidance Service Circle Pines.
Wu, K., Wang, Z., Zhang, Y., Yao, J., & Zhang, Z. (2020). Transcutaneous vagus nerve stimulation for the treatment of drug-resistant epilepsy: A meta-analysis and systematic review. ANZ Journal of Surgery, 90, 467-472. doi: 10.1111/ans.15681
Yakunina, N., Kim, S. S., & Nam, E-C. (2016). Optimization of transcutaneous vagus nerve stimulation using functional MRI. Neuromodulation, 20(3), 290-300. doi: 10.1111/ner.12541
Yakunina, N., Kim, S. S., & Nam, E-C. (2018). BOLD fMRI effects of transcutaneous vagus nerve stimulation in patients with chronic tinnitus. PLoS ONE, 13(11), e0207281. https://doi.org/10.1371/journal.pone.0207281
Ylikoski, J., Lehtimaki, J., Pirvola, U., Makitie, A., Aarnisalo, A., Hyvarinen, P., & Ylikoski, M. (2017). Non-invasive vagus nerve stimulation reduces sympathetic preponderance in patients with tinnitus. Acta Oto-Laryngologica, 137(4), 426-431. http://dx.doi.org/10.1080/00016489.2016.1269197
Yuan, H., & Silberstein, S. D. (2015). Vagus nerve and vagus nerve stimulation, a comprehensive review: Part II. Headache, 56, 259-266. doi: 10.1111/head.12650

### REFERENCES FOR EXAMPLE #3

Badran, B. W., Dowdle, L. T., Mithoefer, O. J., LaBate, N. T., Coatsworth, J., Brown, J. C., DeVries, W. H., Austelle, C. W., McTeague, L. M., & George, M. S. (2018). Neurophysiologic effects of transcutaneous auricular vagus nerve stimulation (taVNS) via electrical stimulation of the tragus: A concurrent taVNS/fMRI study and review. Brain Stimulation, 11, 492-500. https://doi.org/10.1016/j.brs.2017.12.009
Bowey, J. A. (1995). Socioeconomic status differences in preschool phonological sensitivity and first-grade reading achievement. Journal of Educational Psychology, 87(3), 476-487. https://doi.org/10.1037/0022-0663.87.3.476
Bradley, R. H., & Corwyn, R. F. (2002). Socioeconomic status and child development. Annual Review of Psychology, 53, 371-399. doi: 10.1146/annurev.psych.53.100901.135233
Butterfuss, R., & Kendeou, P. (2018). The role of executive functions in reading comprehension. Educational Psychology Review, 30(3), 801-826. https://doi org/10.1007/s10648-017-9422-6
Cancer, A., & Antonietti, A. (2018). tDCS modulatory effect on reading processes: A review of studies on typical readers and individuals with dyslexia. Frontiers in Behavioral Neuroscience, 12, 162. doi: 10.3389/fnbeh.2018.00162
Centanni, T. M., King, L. W., Eddy, M. D., Whitfield-Gabrieli, S., & Gabrieli, J. D. E. (2017). Development of sensitivity versus specificity for print in the visual word form area. Brain & Language, 170, 62-70. http://dx.doi.org/10.1016/j.band1.2017.03.009
Cheng, Y., & Wu, X. (2017). The relationship between SES and reading comprehension in Chinese: A mediation model. Frontiers in Psychology, 8, 672. https://doi.org/10.3389/fpsyg.2017.00672
Christodoulou, J. A., Del Tufo, S. N., Lymberis, J., Saxler, P. K., Ghosh, S. S., Triantafyllou, C., Whitfield-Gabrieli, S., & Gabrieli, J. D. E. (2014). Brain bases of reading fluency in typical reading and impaired fluency in dyslexia. PLoS ONE, 9(7), e100552. https://doi.org/10.1371/journal.pone.0100552
Clark, K. B., Naritoku, D. K., Smith, D. C., Browning, R. A., & Jensen, R. A. (1999). Enhanced recognition memory following vagus nerve stimulation in human subjects. Nature Neuroscience, 2(1), 94-98. https://doi.org/10.1038/4600
Clarke, P. J., Snowling, M. J., Truelove, E., & Hulme, C. (2009). Ameliorating children's reading-comprehension difficulties: A randomized controlled trial. Psychological Science, 21(8), 1106-1116. https://doi.org/10.1177/0956797610375449
Costanzo, F., Menghini, D., Caltagirone, C., Oliveri, M., & Vicari, S. (2012). High frequency rTMS over the left parietal lobule increases non-word reading accuracy. Neuropsychologia, 50, 2645-2651. http://dx.doi.org/10.1016/j.neuropsychologica.2012.07.017
Costanzo, F., Menghini, D., Caltagirone, C., Oliveri, M., & Vicari, S. (2013). How to improve reading skills in dyslexics: The effect of high frequency rTMS. Neuropsychologia, 51, 2953-2959. http://dx.doi.org/10.1016/j.neuropsychologia.2013.10.D18
Costanzo, F., Varuzza, C., Rossi, S., Sdoia, S., Varvara, P., Oliveri, M., Koch, G., Vicari, S., & Menghini, D. (2016a). Reading changes in children and adolescents with dyslexia after transcranial direct current stimulation. NeuroReport, 27, 295-300. https://doi.org/10.1097/WNR.0000000000000536
Costanzo, F., Varuzza, C., Rossi, S., Sdoia, S., Varvara, P., Oliveri, M., Giacomo, K., Vicari, S., & Menghini, D. (2016b). Evidence for reading improvement following tDCS treatment in children and adolescents with dyslexia. Restorative Neurology and Neuroscience, 34, 215-226. https://doi.org/10.3233/RNN-150561
Daneman, M., & Carpenter, P. A. (1980). Individual differences in working memory and reading. Journal of Verbal Learning and Verbal Behavior, 19, 450-466. https://doi.org/10.1016/S0022-5371(80)90312-6
De Ridder, D., Vanneste, S., Engineer, N. D., & Kilgard, M. P. (2013). Safety and efficacy of vagus nerve stimulation paired with tones for the treatment of tinnitus: A case series. Neuromodulation, 17, 170-179. https://doi.org/10.1111/ner.12127
Engineer, N. D., Riley, J. R., Seale, J. D., Vrana, W. A., Shetake, J. A., Sudanagunta, S. P., Borland, M. S., & Kilgard, M. P. (2011). Reversing pathological neural activity using targeted plasticity. Nature, 470, 101-106. https://doi.org/10.1038/nature09656
Faul, F., Erdfelder, E., Lang, A-G., & Buchner, A. (2007). G*Power 3: A flexible statistical power analysis program for the social, behavioral, and biomedical sciences. Behavior Research Methods, 39(2), 175-191.
Florit, E., & Cain, K. (2011). The simple view of reading: Is it valid for different types of alphabetic orthographies? Educational Psychology Review, 23, 553-573. https://doi.org/10.1007/s10648-011-9175-6
Follmer, D. J. (2018). Executive function and reading comprehension: A meta-analytic review. Educational Psychologist, 53(1), 42-60. https://doi.org/10.1080/00461520.2017.1309295
Frangos, E., Ellrich, J., & Komisaruk, B. R. (2015). Non-invasive access to the vagus nerve central projections via electrical stimulation of the external ear: fMRI evidence in humans. Brain Stimulation, 8(3), 624-636. https://doi.org/10.1016/j.brs.2014.11.018
Gough, P. B., & Tunmer, W. E. (1986). Decoding, reading, and reading disability. Remedial and Special Education, 7(1), 6-10. https://doi.org/10.1177/074193258600700104
Hoover, W. A., & Gough, P. B. (1990). The simple view of reading. Reading and Writing: An Interdisciplinary Journal, 2, 127-160. https://doi org/10.1007/BF00401799
Hulsey, D. R., Shedd, C. M., Sarker, S. F., Kilgard, M. P., & Hays, S. A. (2019). Norepinephrine and serotonin are required for vagus nerve stimulation directed cortical plasticity. Experimental Neurology, 320, 112975. https://doi.org/10.1016/j.expneurol.2019.112975
Jacobs, H. I. L., Riphagen, J. M., Razat, C. M., Wiese, S., & Sack, A. T. (2015). Transcutaneous vagus nerve stimulation boosts associative memory in older individuals. Neurobiology of Aging, 36, 1860-1867. http://dx.doi.org/10.1016/j.neurobiolaging.2015.02.023
Kaufman, A. S. & Kaufman, N. L. (2004). K-BIT: Kaufman brief intelligence test. (2nd ed.). Bloomington, Minn.: Pearson.
Khodaparast, N., Hays, S. A., Sloan, A. M., Hulsey, D. R., Ruiz, A., Pantoja, R. L., Rennaker, R. L., & Kilgard, M. P. (2013). Vagus nerve stimulation during rehabilitative training improves forelimb strength following ischemic stroke. Neurobiology of Disease, 60, 80-88. https://doi.org/10.1161/STROKEAHA.114.006654
Kieffer, M. J. (2010). Socioeconomic status, English proficiency, and late-emerging reading difficulties. Educational Researcher, 39(6), 484-486. https://doi.org/10.3102/0013189X10378400
Klomjai, W., Katz, R., & Lackmy-Vallee, A. (2015). Basic principles of transcranial magnetic stimulation (TMS) and repetitive TMS (rTMS). Annals of Physical and Rehabilitation Medicine, 58, 208-213. https://doi.org/10.1016/j.rehab.2015.05.005
LARRC. (2015). Learning to read: Should we keep things simple?. Reading Research Quarterly, 50(2), 151-169. https://doi.org/10.1002/rrq.99
LARRC, & Chiu, Y. D. (2018). The simple view of reading across development: Prediction of grade 3 reading comprehension from prekindergarten skills. Remedial and Special Education, 39(5), 289-303. https://doi.org/10.1177/0741932518762055
LARRC, Pratt, A., & Logan, J. (2014). Improving language-focused comprehension instruction in primary-grade classrooms: Impacts of the Let's Know! experimental curriculum. Educational Psychology Review, 26, 357-377. https://doi.org/10.1044/2019_JSLHR-L-19-0015
Lehtimaki, J., Hyvarinen, P., Ylikoski, M., Bergholm, M., Makela, J. P., Aarnisalo, A., Pirvola, U., Makitie, A., & Ylikoski, J. (2013). Transcutaneous vagus nerve stimulation in tinnitus: A pilot study. Acta Oto-Laryngologica, 133, 378-382. https://doi.org/10.3109/00016489.2012.750736
Llanos, F., McHaney, J. R., Schuerman, W. L., Yi, H. G., Leonard, M. K., & Chandrasekaran, B. (2020). Non-invasive peripheral nerve stimulation selectively enhances speech category learning in adults. NPJ Science of Learning, 5, 12. https://doi.org/10.1038/s41539-020-0070-0
Martins, L. Z., & Carnio, M. S. (2020). Reading comprehension in dyslexic schoolchildren after an intervention program. CoDAS, 32(1), e20180156. https://doi.org/10.1590/2317-1782/20192018156
McKee, S. (2012). Reading comprehension, what we know: A review of research 1995 to 2011. Language Testing in Asia, 2(1), 45-58. https://doi.org/10.1186/2229-0443-2-1-45
Medeiros, L. F., Custodio de Souza, I. C., Vidor, L. P., de Souza, A., Deitos, A., Volz, M. S., Fregni, F., Caumo, W., & Tones, I. L. S. (2012). Neurobiological effects of transcranial direct current stimulation: A review. Frontiers in Psychiatry, 3, 110. doi: 10.3389/fpsyt.2012.00110
Mellard, D. F., Fall, E., & Woods, K. L. (2010). A path analysis of reading comprehension for adults with low literacy. Journal of Learning Disabilities, 43(2), 154-165. https://doi.org/10.1177/0022219409359345
O'Brien, B. A., Mansfield, J. S., & Legge, G. E. (2005). The effect of print size on reading speed in dyslexia. Journal of Research in Reading, 28(3), 332-349. https://doi.org/10.1111/j.1467-9817.2005.00273.x
O'Brien, B. A., & Wallot, S. (2016). Silent reading fluency and comprehension in bilingual children. Frontiers in Psychology, 7, 1265. doi: 10.3389/fpsyg.2016.01265
Oliver, R., & Young, S. (2016). Improving reading fluency and comprehension in adult ESL learners using bottom-up and top-down vocabulary training. Studies in Second Language Learning and Teaching, 6(1), 111-133. https://doi.org/10.14746/ssllt.2016.6.1.6
Peirce, J., Gray, J. R., Simpson, S., MacAskill, M., Hochenberger, R., Sogo, H., Kastman, E., & Lindelov, J. K. (2019). PsychoPy2: Experiments in behavior made easy. Behavior Research Methods, 51(1), 195-203. https://doi.org/10.3758/s13428-018-01193-y
Peng, P., Barnes, M., Wang, C., Wang, W., Li, S., Swanson, H. L., Dardick, W., & Tao, S. (2018). A meta-analysis on the relation between reading and working memory. Psychological Bulletin, 144(1), 48-76. http://dx.doi.org/10.1037/bul0000124
Pennington, B. F., & Bishop, D. V. M. (2009). Relations among speech, language, and reading disorders. Annual Review of Psychology, 60, 283-306. https://doi.org/10.1146/annurev.psych.60.110707.163548
Picciotto, M. R., Higley, M. J., & Mineur, Y. S. (2012). Acetylcholine as a neuromodulator: Cholinergic signaling shapes nervous systems function and behavior. Neuron, 76(1), 116-129. https://doi.org/10.1016/j.neuron.2012.08.036
Porter, B. A., Khodaparast, N., Fayyaz, T., Cheung, R. J., Ahmed, S. S., Vrana, W. A., Rennaker, R. L., Jr., & Kilgard, M. P. (2012). Repeatedly pairing vagus nerve stimulation with a movement reorganizes primary motor cortex. Cerebral Cortex, 22(10), 2365-2374. https://doi.org/10.1093/cercor/bhr316
Redgrave, J. N., Moore, L., Oyekunle, T., Ebrahim, M., Falidas, K., Snowdon, N., Ali, A., & Majid, A. (2018). Transcutaneous auricular vagus nerve stimulation with concurrent upper limb repetitive task practice for poststroke motor recovery: A pilot study. Journal of Stroke and Cerebrovascular Diseases, 27(7), 1998-2005. https://doi.org/10.1016/j.jstrokecerebrovasdis.2018.02.056
Sabatini, J. P., Shore, J., Holtzman, S., & Scarborough, H. S. (2011). Relative effectiveness of reading intervention programs for adults with low literacy. Journal of Research on Educational Effectiveness, 4, 118-133. https://doi.org/10.1080/19345747.2011.555290
Sackeim, H. A., Keilp, J. G., Rush, A. J., George, M. S., Marangell, L. B., Dormer, J. S., Burt, T., Lisanby, S. H., Husain, M., Cullum, C. M., Oliver, N., & Zboyan, H. (2001). The effects of vagus nerve stimulation on cognitive performance in patients with treatment-resistant depression. Neuropsychiatry, Neuropsychology, and Behavioral Neurology, 14(1), 53-62.
Sheslow, D. & Adams, W. (2009). Wide range assessment of memory and learning. (2nd ed.). Austin, Tex.: ProEd, Inc.
Simmons, F., & Singleton, C. (2000). The reading comprehension abilities of dyslexic students in higher education. Dyslexia, 6, 178-192. https://doi.org/10.1002/1099-0909(200007/09)6:3<178::AID-DYS171>3.0.CO; 2-9
Spencer, M., Richmond, M. C., & Cutting, L. E. (2020). Considering the role of executive function in reading comprehension: A structural equation modeling approach. Scientific Studies of Reading, 24(3), 179-199. https://doi.org/10.1080/10888438.2019.1643868
Sun, L., Perakyla, J., Holm, K., Haapasalo, J., Lehtimaki, K., Ogawa, K. H., Peltola, J., & Hartikainen, K. M. (2017). Vagus nerve stimulation improves working memory performance. Journal of Clinical and Experimental Neuropsychology, 39(10), 954-964. https://doi.org/10.1080/13803395.2017.1285869
Thakkar, V. J., Engelhart, A. S., Khodaparast, N., Abadzi, H., & Centanni, T. M. (2020). Transcutaneous auricular vagus nerve stimulation enhances learning of novel letter-sound relationships in adults. Brain Stimulation, 13(6), 1813-1820. https://doi.org/10.1016/j.brs.2020.10.012
Torgesen, J., Wagner, R., & Rashotte, C. (2012). Test of word reading efficiency (2nd ed.). Austin, Tex.: ProEd, Inc.
Torppa, M., Georgious, G. K., Lerkkanen, M-K., Niemi, P., Poikkeus, A-M., & Nurmi, J-E. (2016). Examining the simple view of reading in a transparent orthography: A longitudinal study from kindergarten to grade 3. Merrill-Palmer Quarterly, 62(2), 179-206. https://doi.org/10.13110/merrpalmquar1982.62.2.0179
Tully, K., & Volshakov, V. Y. (2010). Emotional enhancement of memory: How norepinephrine enables synaptic plasticity. Molecular Brain, 3, 15. https://doi.org/10.1186/1756-6606-3-15
Veeravagu, J., Muthusamy, C., Marimuthu, R., & Michael, A. S. (2010). Using Bloom's taxonomy to gauge students' reading comprehension performance. Canadian Social Science, 6(3), 205-212. doi: 10.3968/J.CSS.1923669720100603.023
Wagner, R. K., Torgesen, J. K., & Rashotte, C. A. (2013). CTOPP-2: Comprehensive test of phonological processing. (2nd ed.). Austin, Tex.: ProEd, Inc.
Wolf, M., & Obregon, M. (1992). Early naming deficits, developmental dyslexia, and a specific deficit hypothesis. Brain and Language, 42, 219-247. https://doi.org/10.1016/0093-934X(92)90099-Z
Woodcock, R. W. (2011). Woodcock reading mastery tests. (3rd ed.). Bloomington, Minn.: American Guidance Service Circle Pines.
Wiederholt, J. L. & Bryant, B. R. (2012). Gray oral reading tests. (5th ed.). Austin, Tex.: ProEd, Inc.
Yakunina, N., Kim, S. S., & Nam, E-C. (2016). Optimization of transcutaneous vagus nerve stimulation using functional Mill. Neuromodulation, 20(3), 290-300. https://doi.org/10.1111/ner. 12541
Yakunina, N., Kim, S. S., & Nam, E-C. (2018). BOLD fMRI effects of transcutaneous vagus nerve stimulation in patients with chronic tinnitus. PLoS ONE, 13(11), e0207281. doi: https://doi.org/10.1371/journal.pone.0207281

What is claimed is:

1. A method of enhancing language learning, the method comprising the steps of:
positioning a non-invasive transcutaneous auricular vagus nerve stimulation device in or on the ear of a human subject; and
conducting learning exercises while periodically stimulating the vagus nerve with the device;
wherein the learning exercises are selected from the group consisting of the learning of letter-sound relationships, the learning of memory-based reading comprehension, and learning retention of a novel language.

2. The method of claim 1, wherein the intensity of the stimulation is varied during the learning exercise.

3. The method of claim 1, wherein the frequency of the stimulation administered by the device is at least 5 Hz.

4. The method of claim 1, wherein the frequency is varied depending upon the task involved in the learning exercise.

5. The method of claim 1, wherein the position of the device in the human ear is varied.

6. The method of claim 1, wherein the learning exercise is letter-sound relationships and the front size and angle of the letters are varied during the learning exercises.

7. The method of claim 1, wherein the stimulation is in the form of a series of pulses with the current applied as a square biphasic pulse at 5 Hz or greater frequency and at least a 200 ms pulse width.

8. The method of claim 1, wherein the stimulation is applied to the left posterior tragus region of the human ear.

9. The method of claim 1, wherein the stimulation is applied to the left cymba conchae region of the human ear.
